(19) United States
(12) Patent Application Publication
(10) Pub. No.: US 2020/0318140 A1
(43) Pub. Date: Oct. 8, 2020

(54) METHODS AND COMPOSITIONS FOR INCREASING RNA ACTIVITY IN A CELL

(71) Applicants: Bioverativ Therapeutics, Inc., Waltham, MA (US); MaxCyte, Inc., Gaithersburg, MD (US); Sangamo Therapeutics, Inc., Richmond, CA (US)

(72) Inventors: Siyuan Tan, Cambridge, MA (US); Dale Ando, Richmond, CA (US); Andreas Reik, Richmond, CA (US); Linhong Li, Gaithersburg, MD (US); Madhusudan V. Peshwa, Gaithersburg, MD (US); Haiyan Jiang, Cambridge, MA (US)

(21) Appl. No.: 16/839,947

(22) Filed: Apr. 3, 2020

Related U.S. Application Data
(63) Continuation of application No. 15/347,182, filed on Nov. 9, 2016, now abandoned.
(60) Provisional application No. 62/254,900, filed on Nov. 13, 2015.

(51) Int. Cl. C12N 15/90; A61K 48/00; C12N 15/87

(52) U.S. Cl. CPC C12N 15/907; C12N 15/87; A61K 48/005

(57) ABSTRACT

Disclosed herein are methods and compositions for increasing RNA activity in a cell.

### CROSS-REFERENCE TO RELATED APPLICATIONS

The present application is a continuation of U.S. patent application Ser. No. 15/347,182, filed Nov. 9, 2016, which claims the benefit of U.S. Provisional Patent Application No. 62/254,900, filed Nov. 13, 2015, the disclosure of which is hereby incorporated by reference in its entirety.

### STATEMENT OF RIGHTS TO INVENTIONS MADE UNDER FEDERALLY SPONSORED RESEARCH

Not applicable.

### TECHNICAL FIELD

The present disclosure is in the field of genome engineering, particularly increasing RNA activity in a cell.

### BACKGROUND

One of the most promising approaches in biological research, including in the gene therapy of a large number of diseases, involves the use of in vitro genetic modification of stem cells followed by transplantation and engraftment of the modified cells in a patient. Particularly promising is when the introduced stem cells display long term persistence and multi-lineage differentiation. Hematopoietic stem cells, most commonly in the form of cells enriched based on the expression of the CD34 cell surface marker, are a particularly useful cell population since they can be easily obtained and contain the long term hematopoietic stem cells (LT-HSCs), which can reconstitute the entire hematopoietic lineage after transplantation.

Various methods and compositions for targeted cleavage of genomic DNA have been described. Such targeted cleavage events can be used, for example, to induce targeted mutagenesis, induce targeted deletions of cellular DNA sequences, and facilitate targeted recombination at a predetermined chromosomal locus in cells from any organism. Cleavage can occur through the use of specific nucleases such as engineered zinc finger nucleases (ZFN), transcription-activator like effector nucleases (TALENs), using the CRISPR/Cas system with an engineered crRNA/tracr RNA (single guide RNA or sgRNA') to guide specific cleavage and/or using the Argonaute system (e.g., from T thermophilus, known as ‘TtAgo’. See, e.g., U.S. Pat. Nos. 9,255,250; 9,200,266; 9,045,763; 9,005,973; 9,150,847; 8,956,828; 8,945,868; 8,703,489; 8,586,526; 6,534,261; 6,599,692; 6,503,717; 6,689,558; 7,067,317; 7,262,054; 7,888,121; 7,972,854; 7,914,796; 7,951,925; 8,110,379; 8,409,861; U.S. Patent Publications 20030232410; 20050208489; 20050026157; 20050064474; 20060063231; 20080159996; 201000218264; 20120017290; 20110265198; 20130137104; 20130122591; 20130177983; 20130196373; 20140120622; 20150056705; 20150335708; 20160030477 and 20160024474; Swarts et al (2014) Nature 507(7491): 258-261, the disclosures of which are incorporated by reference in their entireties for all purposes. These methods often involve the use of engineered cleavage systems to induce a double strand break (DSB) or a nick (single-stranded break) in a target DNA sequence such that repair of the break by an error born process such as non-homologous end joining (NHEJ) or repair using a repair template (homology directed repair or HDR) can result in the knock out of a gene or the insertion of a sequence of interest (targeted integration). The repair pathway followed (NHEJ versus HDR or both) typically depends on the presence of a repair template and the activity of several competing repair pathways. However, hematopoietic stem cells have proven to be more difficult to modify than many other cell types.

Typically, the introduction of mRNA into cells (somatic cells) has been accomplished at between about room temperature and the optimal growth temperature of the cells (e.g., 37° C. for mammalian cells). See, e.g., U.S. Patent Publication No.

20110236978. In addition, U.S. Pat. No. 8,772,008 describes methods for increasing nuclease activity involve introducing a polynucleotide encoding one or more nucleases into a cell and then culturing the cells at reduced temperatures (e.g., 27° C. and 33° C.) for between 1 and 4 days.

Nonetheless, there remains a need for compositions and methods for increasing RNA activity in a cell, particularly a hematopoietic stem cell or progenitor stem cell.

### SUMMARY

Described herein are methods and compositions to transiently introduce and/or express a polynucleotide in a cell, preferably an RNA (e.g., mRNA). Specifically, the invention describes methods for contacting a cell with RNA such that the RNA is taken up by the cell, and is active. The methods and compositions of the invention are advantageous over the standard methods in the art because the activity of the RNAs used in the invention is more effective than previous methods.

In one aspect, provided herein is a method for modifying a cell (e.g., hematopoietic stem cell or progenitor stem cell) or cells (e.g., a population of cells in a cell culture), the method comprising: (a) cooling the cell(s) in a vessel to about 15° C. or below; and (b) introducing an exogenous polynucleotide (e.g., RNA) into the cooled cells under conditions such that the cell is modified. In certain embodiments, the method further comprises the step of cooling the cell(s) (e.g., the vessel containing the cell(s)) to about 15° C. or below. In still further embodiments, the method comprises the step of contacting the cooled vessel and the cells to cool the cell(s). Any of the methods described herein may further comprise the step of cooling the exogenous polynucleotide (e.g., RNA) to about 15° C. or below and/or combining the cooled exogenous polynucleotide (e.g., RNA) and cells within the vessel and maintaining the temperature at about 15° C. or below (e.g., at a temperature of no more than 15° C. and/or no less than −15° C.). Furthermore, in certain embodiments, the method further comprises the step of placing the vessel in a device (e.g., an electroporation device) suitable for introducing exogenous polynucleotide (e.g., RNA) into the cells. All or at least a part of the device (e.g., the part that operably contacts the vessel containing the cells) may be cooled to about 15° C. or below prior to placing the vessel in the device. Any of the methods described herein may further comprise the step of incubating the cells under conditions suitable for expressing the polynucleotide (e.g., RNA) in the cell(s), thereby modifying the cell(s). The exogenous RNA may include any suitable RNA such as mRNA, rRNA, tRNA, siRNA, sgRNA, RNAi and/or miRNA including combinations thereof.

In any of the methods described herein, the exogenous polynucleotide (e.g., mRNA) may encode a heterologous nuclease or component thereof. In certain embodiments, the methods further comprise introducing a donor polynucleotide into the cell comprising the heterologous nuclease such that the donor polynucleotide is integrated into the genome of the cell. The nuclease may be a zinc finger nuclease (ZFN), a TALE-effector domain nuclease (TALEN) and/or a CRISPR/Cas nuclease system. Furthermore, and in embodiments in which the polynucleotide (e.g., RNA) encodes a ZFN, the methods may further comprise (c) culturing the cells of step (b) (e.g., cells comprising an exogenous nuclease and/or donor polynucleotide) at about 27° C. to about 33° C.; and (d) culturing the cells of step (c) at an optimal growth temperature.

In another aspect, the invention features a cell (or population of cells) that are modified by one or a combination of methods as described herein. As will be apparent from the disclosure and Examples that follow, the invention is flexible and is not limited to the use of any particular polynucleotide (e.g., those encoding a ZFN, CRISPR/Cas, TALEN and/or other polypeptide) so long as intended results are achieved.

Also described herein are methods and compositions for expressing an exogenous nuclease in the cell and for genomically modifying that cell using an exogenous nuclease (e.g., ZFN, TALEN, CRISPR/Cas nuclease system). The methods involve introducing polynucleotides (e.g., RNA) encoding an exogenous nuclease (e.g., ZFN or TALEN) or polynucleotides that are a component of a nuclease system (e.g., single guide RNAs of the CRISPR/Cas system) into the cell at temperatures below about room temperature (below about 15° C. and preferably above about -15° C.), for example using an ice bath or other device adapted for cooling, and maintaining the cells, polynucleotides and cell-polynucleotide mixture at the reduced temperature for a period of time. The methods and compositions result in genetic modifications to the cell(s) (e.g., through introduction of mutations via NHEJ and/or targeted donor nucleic acid insertion via homologous recombination or non-homologous methods). The methods and compositions described herein provide important advantages including significantly increasing the activity of the polynucleotide within the cells, for example, by increasing the efficiency of nuclease activity in the cell (e.g., hematopoietic stem cells and progenitor stem cells) as compared to methods in which the cells (and/or nucleases and/or donors) are not cooled.

In another aspect, described herein is a method for genetically modifying one or more cells, the method comprising cooling the cells (e.g., in a vessel, for example a population of cells in a solution such as a culture medium) to less than about 15° C. and more particularly subjecting the cell to severe cold-shock conditions (defined below); and introducing an exogenous nuclease into the cooled cells such that the genome of the cell is modified. In certain embodiments, the methods further comprise introducing a donor sequence into the cell such that the donor sequence is integrated into the genome of the cell. In any of the methods described herein, the cells, polynucleotides (e.g., RNAs), exogenous nuclease(s) and/or optional donors may be subject to the extreme cold-shock conditions before, during and/or after introduction of the nucleases into the cell. In certain embodiments, the cells, polynucleotides (e.g., RNAs), exogenous nuclease and/or cell-nuclease mixture are all cooled accordingly severe cold-shock conditions before, during and after introduction of the exogenous nuclease into the cells. In certain embodiments, the cooling to the extreme cold-shock temperature is a temperature of between about −15° C. to about 15° C. (including any temperature therebetween, such as −15° C., −14° C., −13° C., −12° C., −11° C., −10° C., −9° C., −8° C., −7° C., −6° C., −5° C., −4° C., −3° C., −2° C., −1° C., 0° C., 1° C., 2° C., 3° C., 4° C., 5° C., 6° C., 7° C., 8° C., 9° C., 10° C., 11° C., 12° C., 13° C., 14° C., 15° C.) for example using an ice bath and/or refrigeration to maintain a temperature of between about 0° C. and about 4° C. In certain embodiments, the extreme cold-shock temperature is no more than 20° C. or no more than 15° C. and/or no less than −20° C. or no less than −15° C., including, for example any temperature between about −4° C. and about 4° C. In certain embodiments, the cells, polynucleotides (e.g., RNAs), nucleases and/or donors are cooled to the subject to severe cold-shock conditions during introduction (e.g., transfection) of the nucleases and/or donors. In other embodiments, the cells, RNAs, nucleases and/or donors are cooled subjected to the severe cold-shock conditions during and after introduction of the nucleases and/or donors (e.g., during transfection and then the cell-nuclease mixture is held at the severe cold-shock conditions for a period of time.

In still further embodiments, the components (cells, polynucleotides such as RNAs, nucleases and/or donors) are individually subjected to the severe cold-shock conditions before introduction of the nucleases and/or donors into the cells and held at these conditions during and/or after introduction.

In another aspect, there is provided herein a method for expressing a nuclease in a cell, the method comprising the steps of: (a) cooling the cell to below room temperature (e.g., subjecting the cell to severe cold-shock conditions); (b) contacting the cell with a polynucleotide encoding the nuclease such that the polynucleotide is introduced and expressed in the cell. Also provided is a method for expressing a nuclease in a cell, the method comprising the steps of: (a) cooling the cell to below room temperature (e.g., subjecting the cell to severe cold-shock conditions); (b) contacting the cell with a nuclease comprising a single guide RNA (sgRNA) and at least one cleavage domain such that the sgRNA and cleavage domain (e.g., CAS and/or FokI endonuclease) associate to cleave the genome of the cell. In certain embodiments, the cells are hematopoietic stems cells and progenitor stem cells optionally obtained from bone marrow, particularly (BM)-derived CD34+ cells taken from a subject (e.g., a patient). In any of the methods described herein, the polynucleotide can be introduced into the cell via any suitable method, including electroporation.

In any of the methods described herein, the nuclease(s) may be delivered to the cell in mRNA form. The optional donors, typically delivered to the cell in DNA form, may also be delivered to the cell in RNA form (e.g., mRNA). In one aspect, the invention provides a cell described herein at about 15° C. or lower (e.g., −15° C. to about 15° C., including −5° C. to about 15° C. or 0° C. to about 4° C. or 0° C. to about 15° C.) comprising a nuclease and a donor nucleic acid such that the nuclease mediates targeted integration of the exogenous sequence into the genome. In certain embodiments, the cell is a eukaryotic cell (e.g., a mammalian cell), for example a stem cell (e.g., hematopoietic stem cell such as a CD34+ stem cell). In some aspects, the host cells are an established cell line while in other aspects, the host cell is a primary cell isolated from a mammal. In some aspects, the invention provides a cell as described above wherein the donor nucleic acid encodes a reporter construct which may be transiently or stably expressed in the cell. Any of the cells may further comprise a sequence encoding a nuclease or a component of a nuclease (e.g., sgRNA of CRISPR/Cas system).

In yet another aspect, provided herein is a method of genetically modifying the cell, the method comprising the steps of: introducing one or more polynucleotides encoding or comprising components of the nuclease(s) into any of the cells described herein under severe cold shock conditions; culturing the cells at a temperature less than the optimal growth temperature (e.g., 27° C. to 33° C. for mammalian cells) for a period of time (e.g., overnight to days); culturing the cells at an optimal growth temperature (e.g., 37° C. for mammalian cells) such that the cell is genetically modified. In certain embodiments, the methods further comprise the step of determining the level of nuclease activity.

In any of the methods described herein, the nuclease may comprise, for example, a non-naturally occurring DNA-binding domain (e.g., an engineered zinc finger protein, a TAL-effector nuclease fusion protein, or an engineered DNA-binding domain from a homing endonuclease, single guide RNA). In certain embodiments, the nuclease is a zinc finger nuclease (ZFN) or pair of ZFNs. In other embodiments, the nuclease is a TAL-effector domain nuclease fusion protein. In still further embodiments, the nuclease is a CRISPR/Cas nuclease system.

Any of the methods may further comprise introducing an exogenous sequence into the cell such that the nuclease(s) mediate(s) targeted integration of the exogenous sequence into the genome. In certain embodiments, the exogenous sequence is introduced at the same time as the nuclease(s) and the exogenous sequence is also subject to severe cold shock prior to introduction into the cell. In some aspects, the exogenous sequence may comprise a sequence encoding a protein (e.g., a therapeutic protein and/or a reporter). In certain embodiments, the methods further comprising isolating the cells expressing the exogenous sequence. In any of the methods described herein, the genomic modification is a gene disruption and/or a gene addition. The exogenous sequence may be introduced by any suitable method, including electroporation and may be introduced by the same or different methods than the nuclease(s).

In another aspect, described herein is a genetically modified cell (e.g., stem cells as described herein) or cell line made by the methods described herein. Non-limiting examples of stem cells include hematopoietic stem cells such as bone marrow (BM)-derived hematopoietic stem and progenitor cells (e.g., CD34+ cells). These cells can be taken from a subject (including a human patient) and maintained ex vivo using known methods. Partially or fully differentiated cells descended from the modified stem cells as described herein are also provided. Compositions such as pharmaceutical compositions comprising the genetically modified cells as described herein are also provided.

In still further aspects, the invention provides methods and compositions for delivering a pulse of mRNA for the induction of polypeptide expression or delivery of a pulse of RNAi or shRNA to the cells described herein with increased efficiency. The methods comprise subjecting the cells and/or RNA to extreme cold shock conditions as described herein during pulsing. In some aspects, the target of the pulsed protein expression plays a role in a DNA repair process, such that when delivered with a suitable nuclease and donor, the pulse of protein expression will skew the DNA repair process towards HDR rather than NHEJ. In other aspects, the target of the RNAi is the expression of a protein that plays a role in the DNA repair process such that an endogenous protein is inhibited, so that when the RNAi is co-delivered with a mRNAs encoding a suitable nuclease along with a donor the DNA, the repair process is skewed towards HDR.

Examples of target proteins include DNA-dependent-protein kinase catalytic subunit (DNA-PKcs) and/or Poly-(ADP-ribose) polymerase 1/2 (PARP1/2). Other suitable targets include PARP1, Ku70/80, DNA-PKcs, XRCC4/XLF, Ligase IV, Ligase III, XRCC1, Artemis and/or Polynucleotide Kinase (PNK). See WO2014/130955, incorporated here by reference.

In some embodiments, the protein expressed from the mRNA pulse transiently locates on to the cell surface and aids in engraftment of the stem cells during a bone marrow transplant. Examples include expression of the CXCR4 protein (Peled et al (1999) Science 283(5403): 845) and/or CD47 and signal regulatory protein α (SIRPα), also known as SHPS-1/BIT/ CD172a as the CD47-SIRPα interaction is thought to play an important role in the engraftment of hematopoietic stem cells (Murata et al, (2014)1 Biochem 155(6): p. 335).

In other aspects, the pulsed protein aids in proliferation of the stem cells. Examples include stem cell factor (SCF), flt3/flt2 ligand (FL), interleukin (IL)-6, IL-11, IL-12, leukemia inhibitory factor (LIF), granulocyte colony stimulating factor (G-CSF), thrombopoietin (TPO) (see Keike and Nakahata (2002)Biochimica et Biophysica Acta 1592: 313) and other factors known in the art.

In some aspects, the pulsed protein is expressed in the presence of a transposon. The pulsed protein may be a transposase where the mRNA encoding the transposase is co-delivered with a donor vector comprising a transgene flanked by two transposon inverted terminal repeats (ITRs) such that upon translation of the transposase, the transgene is inserted into the genome. In preferred embodiments, the transposon is Sleeping Beauty (see Hackett et al (2005), Adv Genet; 54:189), Tol2 (see Huang et al (2010) Mol Ther 18(10):1803), or PiggyBac (see Wilson et al (2007) Mol Ther 15(1), p. 139).

In some aspects, the pulsed protein or is a stem cell differentiation factor, to cause a stem cell to differentiate towards a specific mature cell type. In other aspects, the RNA is an RNAi and inhibits pathways that inhibit differentiation. In preferred embodiments, the stem cells are driven towards differentiating into a cardiomyocyte through transient expression of factors that inhibit Gsk3 (e.g. insulin, see Cross et al (1995) Nature 378: 785). In other preferred embodiments, the stem cells are driven towards cardiomyocyte differentiation by RNAi-driven inhibition of β-catenin (Lian et al (2012) PNAS USA 109(27) E1848). Stem cells may also be driven towards neuronal cell differentiation using a pulse of growth factor expression by the methods and compositions of the invention. Exemplary growth factors include epidermal growth factor (EGF), vascular endothelial growth factor (VEGF) and hepatocyte growth factor (HGF) (see Bae et al (2011) Yonsei Med J 52(3):401).

In some embodiments, the methods and compositions of the invention are used to deliver miRNAs to the cells described herein by subjecting the cells and/or miRNA to extreme cold-shock conditions as described herein. miRNAs have been shown to influence the differentiation state of a stem cell, so delivery of specific miRNAs by the methods herein may cause specific differentiation. For example, miRNA-124 has been shown to promote differentiation of bone marrow derived stem cells into neuronal cells (Zou et al (2014) Neuro Regen Res 9(12): 1241).

In another aspect, the invention provides kits that are useful for increasing the activity of an introduced RNA in the stem cells described herein. In some embodiments, the invention includes kits for increasing the activity of nucleases (e.g. ZFNs, TAL-effector domain nuclease fusion proteins, and/or CRISPR/Cas nuclease systems). In other embodiments, the invention includes kits for increasing the activity of a polypeptide encoding mRNA, the activity of a RNAi or shRNA, and/or for increasing the activity of a miRNA. The kits typically include one or more RNAs, and optional cells for introducing the RNAs in to, instructions for introducing the RNAs into the cells and cold shocking the cells to increase RNA activity. In some instances, the kits comprise RNAs that encode nucleases that bind to a target site, optional cells containing the target site(s) of the nuclease and instructions for introducing the nucleases into the cells and cold shocking the cells to increase nuclease activity. In certain embodiments, the kits comprise at least one construct with the target gene and a known nuclease capable of cleaving within the target gene. Such kits are useful for optimization of cleavage conditions in a variety of varying cell types. Other kits contemplated by the invention may include a known nuclease capable of cleaving within a known target locus within a genome, and may additionally comprise a donor nucleic acid encoding a reporter gene or other transgene of interest. Such kits are useful for optimization of conditions for donor integration. In such kits, the reporter gene/transgene may be operatively linked to a polyadenylation signal and/or a regulatory element (e.g. a promoter).

### BRIEF DESCRIPTION OF THE DRAWINGS

FIGS. 1A through 1D show FACS analysis of GFP expression in BM-derived CD34+ cells transfected with GFP mRNA under the indicated conditions. Top panels show the gating of live cells as R1 based on forward scatter (FSC-H) and side scatter (SSC-H) pattern. Bottom panels show the GFP positive cells as R2, gated by FL1-H for GFP signal among the live cells (R1) from the top panels. Cells were suspended with Maxcyte EP buffer, mixed with GFP mRNA, and electroporated by MaxCyte apparatus except as shown in FIG. 1A (top and bottom), in which the cells did not undergo the GFP mRNA electroporation (“-EP”) and used as negative control. FIG. 1B (top and bottom) shows cells that were electroporated immediately after mixing with GFP mRNA (“0min”). FIG. 1C (top and bottom) show cells mixed with GFP mRNA, cultured at room temperature for 5 minutes and then electroporated (“5min-Rm”). FIG. 1D (top and bottom) show cells on ice, mixed with cooled GFP mRNA, held on ice for 5 minutes and then electroporated (“5min-ice”). Transfection efficiency is improved when cells, mRNA and cell-mRNA mixture are held on ice (R2, 5min-ice), as compared to the 5 minutes incubation at room temperature (R2 5min-Rm).

FIGS. 2A through 2D show FACS analysis of GFP expression in BM-derived CD34+ cells transfected with GFP mRNA under the indicated conditions. The experiment and data analysis was carried out the same as FIG. 1 except that the cells were washed three times with Maxcyte EP buffer (“EP”) prior to mixing with GFP mRNA. FIG. 2A shows results control cells (no RNA). FIG. 2B shows results at 0 minutes; FIG. 2C shows results at 5 minutes at room temperature; and FIG. 2D shows results at 5 minutes with the cells held on ice. The results confirm the data shown in FIG. 1, namely that transfection efficiency is improved when cells, mRNA and cell-mRNA mixture are held on ice. In addition, multiple washings of the cells improved transfection efficiency.

FIGS. 3A through 3D are graphs showing various characteristics of BM-CD34+ cells that electroporated at 0 minute, (0 min) or 5 minute (5min) after mixing with mRNA at room temperature. Cells were cultured overnight at 30° C. and continued for several days at 37° C. Control cells received no GFP mRNA and no electroporation. FIG. 3A shows the percent viability under the indicated conditions. FIG. 3B shows the number of viable cells under the indicated conditions. FIG. 3C shows the percentage of cells expressing GFP. FIG. 3D shows the mean fluorescent intensities (MFI) of cells under the indicated conditions.

FIGS. 4A through 4D are graphs showing various characteristics of BM-CD34+ cells that previously chilled and incubated with mRNA on ice for indicated times (0 min, 2 min, or 5 min) and then electroporated, cultured overnight at 30° C., followed by culturing at 37° C. for additional days. The control cells received no mRNA and no electroporation. FIG. 4A shows the percent viability under the indicated conditions. FIG. 4B shows the number of viable cells under the indicated conditions. FIG. 4C shows the percentage of cells expressing GFP. FIG. 3D shows the mean fluorescent intensities (MFI) of cells under the indicated conditions.

FIG. 5 shows CRISPR/Cas-mediated gene editing via NHEJ-mediated introduction of indels (insertions and/or deletions) at the AAVS1 locus in BM-CD34+ cells under the indicated conditions as analyzed by Cel-1 assay 3 days after transfection. “-EP” refers to cells not electroporated and used as negative control; “EP at 0min” refers to cells that mixed with AAVS-1 targeted CRISPR/Cas reagents and electroporated immediately; “EP at 7 min Room T” refers to cells that mixed with AAVS-1 targeted

CRISPR/Cas reagents, held at room temperature for 7 minutes, and electroporated; and “EP at 8min Ice” refers to refers to cells that previously chilled on ice, mixed with AAVS-1 targeted CRISPR/Cas reagents, held on ice for 8 minutes, and electroporated. Percentages of gene editing are shown below the lanes that showed gene modifications. As shown, the transfections performed and/or held on ice showed significantly increased gene editing rates.

FIG. 6A and 6B show CRISPR/Cas-mediated gene editing (integration of a 6 nucleotide oligo with a HindIII recognition site) at the AAVS1 locus in BM-CD34+ cells under the indicated conditions. FIG. 6A shows gene integration as determined by HindIII digestion. “-EP” refers to cells not being electroporated and used as negative control; “CRISPR+Oligo” refers to cells electroporated with AAVS-1 targeted CRISPR/Cas reagents and donor; and “GFP mRNA” refers to cells electroporated with GFP mRNA. The percentages of gene editing (integration) are shown below the lane that showed integration. FIG. 6B shows FACS analysis of the cells electroporated with GFP mRNA (+EP), and shows that among the live cells (R1), the transfection efficiency was close to 100% (R2), as compared to the no electroporation control cells (-EP), where the GFP positive cells were not detectable (R2, -EP).

FIG. 7 is a representation of a gel showing the gene editing of BM-CD34+ cells at the Bcl11A locus by zinc finger nucleases (ZFN; ZFN A+ZFN B). Lane 1, control cells receive no ZFN treatment; lane 2, BM-CD34+ cells were chilled on ice, mixed with ice-cold ZFN mRNA, and electroporated; and lane 3, BM-CD34+ cells were mixed with ZFN mRNA at room temperature, and electroporated. Implement of the ice-chilling step (lane 2) improves the gene editing efficiency by 10-fold.

FIG. 8 is a table showing the gene editing of BM-CD34+ cells at the Bcl11A erythroid specific enhancer with zinc finger nuclease A (ZFN A) and B (ZFN B). The procedure involved an ice chilling step, which leads to the gene editing level of 52.1 to 59.1% by ZFN A and ZFN B, respectively.

### DETAILED DESCRIPTION

Described herein are compositions and methods to increase activity of RNAs that are introduced into cells. For example, the invention includes nuclease-mediated genomic modification activity and kits for carrying out the methods as described herein. In particular, the methods use severe cold-shock (under about 15° C., e.g., on ice) for varying length of times before, during and/or after cell transfection with the RNAs. In some embodiments, the RNAs encode one or more nucleases. After the cold shock, the cells are returned to a more appropriate temperature to allow the cells to recover, and/or to initiate or increase cell division. In addition, the compositions and methods described herein can also be used to optimize nuclease cleavage conditions for gene disruption and/or gene addition in the cells described herein. Further, the compositions and methods described can also be used to optimize activity of introduced RNAs, including expression and activity of any polypeptides encoded by these RNAs (e.g. mRNAs), or activity of any RNAi, shRNA, sgRNA or miRNAs introduced.

The biological activity of RNAs introduced into cells, including RNAs that encode nucleases is not always the as efficient as possible, for example due to degradation of the polynucleotide in the culture conditions under which the cell is maintained. Thus, methods which can increase RNA activity can be used to increase the rate of affects these RNAs may have on the cells, for example, genomic modifications as a result of introduction of nuclease-encoding RNAs (insertions and/or deletions) in a variety of cell types.

### General

Practice of the methods, as well as preparation and use of the compositions disclosed herein employ, unless otherwise indicated, conventional techniques in molecular biology, biochemistry, chromatin structure and analysis, computational chemistry, cell culture, recombinant DNA and related fields as are within the skill of the art. These techniques are fully explained in the literature. See, for example, Sambrook et al. MOLECULAR CLONING: A LABORATORY MANUAL, Second edition, Cold Spring Harbor Laboratory Press, 1989 and Third edition, 2001; Ausubel et al., CURRENT PROTOCOLS IN MOLECULAR BIOLOGY, John Wiley & Sons, New York, 1987 and periodic updates; the series METHODS IN ENZYMOLOGY, Academic Press, San Diego; Wolfe, CHROMATIN STRUCTURE AND FUNCTION, Third edition, Academic Press, San Diego, 1998; METHODS IN ENZYMOLOGY, Vol. 304, “Chromatin” (P. M. Wassarman and A. P. Wolffe, eds.), Academic Press, San Diego, 1999; and METHODS IN MOLECULAR BIOLOGY, Vol. 119, “Chromatin Protocols” (P. B. Becker, ed.) Humana Press, Totowa, 1999.

### Definitions

The terms “nucleic acid,” “polynucleotide,” and “oligonucleotide” are used interchangeably and refer to a deoxyribonucleotide or ribonucleotide polymer, in linear or circular conformation, and in either single- or double-stranded form. For the purposes of the present disclosure, these terms are not to be construed as limiting with respect to the length of a polymer. The terms can encompass known analogues of natural nucleotides, as well as nucleotides that are modified in the base, sugar and/or phosphate moieties (e.g., phosphorothioate backbones). In general, an analogue of a particular nucleotide has the same base-pairing specificity; i.e., an analogue of A will base-pair with T.

The terms “polypeptide,” “peptide” and “protein” are used interchangeably to refer to a polymer of amino acid residues. The term also applies to amino acid polymers in which one or more amino acids are chemical analogues or modified derivatives of a corresponding naturally-occurring amino acid.

“Binding” refers to a sequence-specific, non-covalent interaction between macromolecules (e.g., between a protein and a nucleic acid). Not all components of a binding interaction need be sequence-specific (e.g., contacts with phosphate residues in a DNA backbone), as long as the interaction as a whole is sequence-specific. Such interactions are generally characterized by a dissociation constant (K_{d}) of 10⁻⁶ M⁻¹ or lower. “Affinity” refers to the strength of binding: increased binding affinity being correlated with a lower K_{d}

A “binding protein” is a protein that is able to bind non-covalently to another molecule. A binding protein can bind to, for example, a DNA molecule (a DNA-binding protein), an RNA molecule (an RNA-binding protein) and/or a protein molecule (a protein-binding protein). In the case of a protein-binding protein, it can bind to itself (to form homodimers, homotrimers, etc.) and/or it can bind to one or more molecules of a different protein or proteins. A binding protein can have more than one type of binding activity. For example, zinc finger proteins have DNA-binding, RNA-binding and protein-binding activity.

A “zinc finger DNA binding protein” (or binding domain) is a protein, or a domain within a larger protein, that binds DNA in a sequence-specific manner through one or more zinc fingers, which are regions of amino acid sequence within the binding domain whose structure is stabilized through coordination of a zinc ion. The term zinc finger DNA binding protein is often abbreviated as zinc finger protein or ZFP.

A “TALE DNA binding domain” or “TALE” is a polypeptide comprising one or more TALE repeat domains/units. The repeat domains are involved in binding of the TALE to its cognate target DNA sequence. A single “repeat unit” (also referred to as a “repeat”) is typically 33-35 amino acids in length and exhibits at least some sequence homology with other TALE repeat sequences within a naturally occurring TALE protein. See, e.g., U.S. Pat. No. 8,586,526.

A “CRISPR/Cas nuclease system” is a nuclease system comprising a single guide RNA (sgRNA) that binds to a target site in DNA and associates with a functional domain (e.g., cleavage domain or transcriptional regulatory domain) to modify the DNA. See, e.g., U.S. Pat. Nos. 8,697,359 and 8,795,965 and U.S. Patent Publication No. 20150056705.

“TtAgo” is a prokaryotic Argonaute protein thought to be involved in gene silencing. TtAgo is derived from the bacteria Thermus thermophilus. See, e.g., Swarts et al (2014) Nature 507(7491): 258-261, G. Sheng et al., (2013) Proc. Natl. Acad. Sci. U.S.A. 111, 652). A “TtAgo system” is all the components required including, for example, guide DNAs for cleavage by a TtAgo enzyme.

“Recombination” refers to a process of exchange of genetic information between two polynucleotides, including but not limited to, donor capture by non-homologous end joining (NHEJ) and homologous recombination. For the purposes of this disclosure, “homologous recombination (HR)” refers to the specialized form of such exchange that takes place, for example, during repair of double-strand breaks in cells via homology-directed repair mechanisms. This process requires nucleotide sequence homology, uses a “donor” molecule to template repair of a “target” molecule (i.e., the one that experienced the double-strand break), and is variously known as “non-crossover gene conversion” or “short tract gene conversion,” because it leads to the transfer of genetic information from the donor to the target. Without wishing to be bound by any particular theory, such transfer can involve mismatch correction of heteroduplex DNA that forms between the broken target and the donor, and/or “synthesis-dependent strand annealing,” in which the donor is used to resynthesize genetic information that will become part of the target, and/or related processes. Such specialized HR often results in an alteration of the sequence of the target molecule such that part or all of the sequence of the donor polynucleotide is incorporated into the target polynucleotide.

Zinc finger binding domains or TALE DNA binding molecules can be “engineered” to bind to a predetermined nucleotide sequence, for example via engineering (altering one or more amino acids) of the recognition helix region of a naturally occurring zinc finger protein or by engineering the RVDs of a TALE protein. Therefore, engineered zinc finger proteins or TALEs are proteins that are non-naturally occurring. Non-limiting examples of methods for engineering zinc finger proteins or

TALEs are design and selection. A “designed” zinc finger protein or TALE is a protein not occurring in nature whose design/composition results principally from rational criteria. Rational criteria for design include application of substitution rules and computerized algorithms for processing information in a database storing information of existing ZFP designs and binding data. A “selected” zinc finger protein or TALE is a protein not found in nature whose production results primarily from an empirical process such as phage display, interaction trap or hybrid selection. See, for example, U.S. Pat. Nos. 8,586,526; 6,140,081; 6,453,242; 6,746,838; 7,241,573; 6,866,997; 7,241,574 and 6,534,261; see also WO 03/016496. A single guide RNA (sgRNA) for use with a Cas protein in a CRISPR/Cas system can also be considered a DNA binding molecule, and it can be “engineered” and/or “designed” to bind to a predetermined nucleic acid sequence through altering the sgRNA sequence such that it targets a desired sequence on the DNA.

The term “sequence” refers to a nucleotide sequence of any length, which can be DNA or RNA; can be linear, circular or branched and can be either single-stranded or double stranded. The term “donor sequence” refers to a nucleotide sequence that is inserted into a genome. A donor sequence can be of any length, for example between 2 and 10,000 nucleotides in length (or any integer value therebetween or thereabove), preferably between about 100 and 1,000 nucleotides in length (or any integer therebetween), more preferably between about 200 and 500 nucleotides in length.

A “target site” or “target sequence” is a nucleic acid sequence that defines a portion of a nucleic acid to which a binding molecule will bind, provided sufficient conditions for binding exist.

“Cold shock” refers to a shift in temperature wherein cells are placed in a hypothermic environment that is colder than optimal growth temperature. The cold shock temperature will depend on the cell type, in particular the temperature that is optimal for cell division to occur in that cell type. For mammalian cells, cold shock temperatures will typically be, about 33° C., about 32° C., about 31° C., about 30° C., about 29° C., about 28° C. or about 27° C. or lower. The term “extreme” or “severe” cold shock” refers to temperatures lower than about 15° C., typically temperatures between about −15° C. and about 15° C., including temperatures at or near freezing (e.g., between about −10° C. and about 10° C. or any value therebetween) and with temperatures of between about 0° C. and about 4° C. being useful for many applications.

The severe cold-shock conditions before, during and/or after introduction of the polynucleotides (e.g., RNAs) and/or nucleases and/or donors into the cells can be applied for any period of time. In certain embodiments, the severe cold shock conditions (before, after and/or during transfection) are used for a period of between 0 and 60 minutes (or any time therebetween), for example, 1, 2, 3, 4, 5 or more minutes before, during and/or after. Thus, the polynucleotides (e.g., RNAs), nucleases, cells and optional donors may be cooled (subject to extreme cold shock) for 0 to 60 minutes or any time therebetween) before introduction of the polynucleotides (e.g., RNAs), nucleases and/or donors into the cells; 0-60 minutes (or any time therebetween) during introduction of the nucleases and/or donors into the cells; and/or 0-60 minutes (or any time therebetween) after introduction of the nucleases and/or donors into the cells.

In any of the methods described herein, the exogenous nuclease may introduced in polynucleotide form (e.g., a viral vector, a non-viral vector, mRNA) or comprise a polynucleotide that is a component of the nuclease (e.g., a single guide RNA). The genomic modifications include, insertions and/or deletions, including insertions of sequences encoding a protein, shRNA, RNAi, miRNA, etc. Furthermore, in any of the methods described herein, the nuclease activity in the cell is increased as compared to cells not subject to severe cold shock.

An “exogenous” molecule is a molecule that is not normally present in a cell, but can be introduced into a cell by one or more genetic, biochemical or other methods. “Normal presence in the cell” is determined with respect to the particular developmental stage and environmental conditions of the cell. Thus, for example, a molecule that is present only during embryonic development of muscle is an exogenous molecule with respect to an adult muscle cell. Similarly, a molecule induced by heat shock is an exogenous molecule with respect to a non-heat-shocked cell. An exogenous molecule can comprise, for example, a functioning version of a malfunctioning endogenous molecule or a malfunctioning version of a normally-functioning endogenous molecule.

An exogenous molecule can be, among other things, a small molecule, such as is generated by a combinatorial chemistry process, or a macromolecule such as a protein, nucleic acid, carbohydrate, lipid, glycoprotein, lipoprotein, polysaccharide, any modified derivative of the above molecules, or any complex comprising one or more of the above molecules. Nucleic acids include DNA and RNA, can be single- or double-stranded; can be linear, branched or circular; and can be of any length. Nucleic acids include those capable of forming duplexes, as well as triplex-forming nucleic acids. See, for example, U.S. Pat. Nos. 5,176,996 and 5,422,251. Proteins include, but are not limited to, DNA-binding proteins, transcription factors, chromatin remodeling factors, methylated DNA binding proteins, polymerases, methylases, demethylases, acetylases, deacetylases, kinases, phosphatases, integrases, recombinases, ligases, topoisomerases, gyrases and helicases.

An exogenous molecule can be the same type of molecule as an endogenous molecule, e.g., an exogenous protein or nucleic acid. For example, an exogenous nucleic acid can comprise an infecting viral genome, a plasmid or episome introduced into a cell, or a chromosome that is not normally present in the cell. Methods for the introduction of exogenous molecules into cells are known to those of skill in the art and include, but are not limited to, lipid-mediated transfer (i.e., liposomes, including neutral and cationic lipids), electroporation, direct injection, cell fusion, particle bombardment, calcium phosphate co-precipitation, DEAE-dextran-mediated transfer and viral vector-mediated transfer. An exogenous molecule can also be the same type of molecule as an endogenous molecule but derived from a different species than the cell is derived from. For example, a human nucleic acid sequence may be introduced into a cell line originally derived from a mouse or hamster.

By contrast, an “endogenous” molecule is one that is normally present in a particular cell at a particular developmental stage under particular environmental conditions. For example, an endogenous nucleic acid can comprise a chromosome, the genome of a mitochondrion, chloroplast or other organelle, or a naturally-occurring episomal nucleic acid. Additional endogenous molecules can include proteins, for example, transcription factors and enzymes.

A “fusion” molecule is a molecule in which two or more subunit molecules are linked, preferably covalently. The subunit molecules can be the same chemical type of molecule, or can be different chemical types of molecules. Examples of the first type of fusion molecule include, but are not limited to, fusion proteins (for example, a fusion between a ZFP or TALE DNA-binding domain and one or more activation domains) and fusion nucleic acids (for example, a nucleic acid encoding the fusion protein described supra). Examples of the second type of fusion molecule include, but are not limited to, a fusion between a triplex-forming nucleic acid and a polypeptide, and a fusion between a minor groove binder and a nucleic acid. The term also includes systems in which a polynucleotide component associates with a polypeptide component to form a functional molecule (e.g., a CRISPR/Cas system in which a single guide RNA associates with a functional domain to modulate gene expression).

Expression of a fusion protein in a cell can result from delivery of the fusion protein to the cell or by delivery of a polynucleotide encoding the fusion protein to a cell, wherein the polynucleotide is transcribed, and the transcript is translated, to generate the fusion protein. Trans-splicing, polypeptide cleavage and polypeptide ligation can also be involved in expression of a protein in a cell. Methods for polynucleotide and polypeptide delivery to cells are presented elsewhere in this disclosure.

A “multimerization domain” (also referred to as a “dimerization domain” or “protein interaction domain”) is a domain incorporated at the amino, carboxy or amino and carboxy terminal regions of a ZFP TF or TALE TF. These domains allow for multimerization of multiple ZFP TF or TALE TF units such that larger tracts of trinucleotide repeat domains become preferentially bound by multimerized ZFP TFs or

TALE TFs relative to shorter tracts with wild-type numbers of lengths. Examples of multimerization domains include leucine zippers. Multimerization domains may also be regulated by small molecules wherein the multimerization domain assumes a proper conformation to allow for interaction with another multimerization domain only in the presence of a small molecule or external ligand. In this way, exogenous ligands can be used to regulate the activity of these domains.

A “gene,” for the purposes of the present disclosure, includes a DNA region encoding a gene product (see infra), as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions.

“Gene expression” refers to the conversion of the information, contained in a gene, into a gene product. A gene product can be the direct transcriptional product of a gene (e.g., mRNA, tRNA, rRNA, miRNA, antisense RNA, ribozyme, structural RNA or any other type of RNA) or a protein produced by translation of an mRNA. Gene products also include RNAs which are modified, by processes such as capping, polyadenylation, methylation, and editing, and proteins modified by, for example, methylation, acetylation, phosphorylation, ubiquitination, ADP-ribosylation, myristilation, and glycosylation.

“Modulation” of gene expression refers to a change in the activity of a gene. Modulation of expression can include, but is not limited to, gene activation and gene repression. Genome editing (e.g., cleavage, alteration, inactivation, random mutation) can be used to modulate expression. Gene inactivation refers to any reduction in gene expression as compared to a cell that does not include a ZFP, modulating RNA (e.g. miRNA or RNAi) TALE protein or CRISPR/Cas system as described herein. Thus, gene inactivation may be partial or complete.

A “region of interest” is any region of cellular chromatin, such as, for example, a gene or a non-coding sequence within or adjacent to a gene, in which it is desirable to bind an exogenous molecule. Binding can be for the purposes of targeted

DNA cleavage and/or targeted recombination. A region of interest can be present in a chromosome, an episome, an organellar genome (e.g., mitochondrial, chloroplast), or an infecting viral genome, for example. A region of interest can be within the coding region of a gene, within transcribed non-coding regions such as, for example, leader sequences, trailer sequences or introns, or within non-transcribed regions, either upstream or downstream of the coding region. A region of interest can be as small as a single nucleotide pair or up to 2,000 nucleotide pairs in length, or any integral value of nucleotide pairs.

“Eukaryotic” cells include, but are not limited to, fungal cells (such as yeast), plant cells, animal cells, mammalian cells and human cells (e.g., T-cells).

The terms “operative linkage” and “operatively linked” (or “operably linked”) are used interchangeably with reference to a juxtaposition of two or more components (such as sequence elements), in which the components are arranged such that both components function normally and allow the possibility that at least one of the components can mediate a function that is exerted upon at least one of the other components. By way of illustration, a transcriptional regulatory sequence, such as a promoter, is operatively linked to a coding sequence if the transcriptional regulatory sequence controls the level of transcription of the coding sequence in response to the presence or absence of one or more transcriptional regulatory factors. A transcriptional regulatory sequence is generally operatively linked in cis with a coding sequence, but need not be directly adjacent to it. For example, an enhancer is a transcriptional regulatory sequence that is operatively linked to a coding sequence, even though they are not contiguous.

With respect to fusion polypeptides, the term “operatively linked” can refer to the fact that each of the components performs the same function in linkage to the other component as it would if it were not so linked. For example, with respect to a fusion polypeptide in which a ZFP or TALE DNA-binding domain is fused to an activation domain, the ZFP or TALE DNA-binding domain and the activation domain are in operative linkage if, in the fusion polypeptide, the ZFP or TALE DNA-binding domain portion is able to bind its target site and/or its binding site, while the activation domain is able to upregulate gene expression. ZFPs fused to domains capable of regulating gene expression are collectively referred to as “ZFP-TFs” or “zinc finger transcription factors”, while TALEs fused to domains capable of regulating gene expression are collectively referred to as “TALE-TFs” or “TALE transcription factors.” When a fusion polypeptide in which a ZFP DNA-binding domain is fused to a cleavage domain (a “ZFN” or “zinc finger nuclease”), the ZFP DNA-binding domain and the cleavage domain are in operative linkage if, in the fusion polypeptide, the ZFP DNA-binding domain portion is able to bind its target site and/or its binding site, while the cleavage domain is able to cleave DNA in the vicinity of the target site. When a fusion polypeptide in which a TALE DNA-binding domain is fused to a cleavage domain (a “TALEN” or “TALE nuclease”), the TALE DNA-binding domain and the cleavage domain are in operative linkage if, in the fusion polypeptide, the TALE DNA-binding domain portion is able to bind its target site and/or its binding site, while the cleavage domain is able to cleave DNA in the vicinity of the target site. With respect to a fusion polypeptide in which a Cas DNA-binding domain is fused to an activation domain, the Cas DNA-binding domain and the activation domain are in operative linkage if, in the fusion polypeptide, the Cas DNA-binding domain portion is able to bind its target site and/or its binding site, while the activation domain is able to up-regulate gene expression. When a fusion polypeptide in which a Cas DNA-binding domain is fused to a cleavage domain, the Cas DNA-binding domain and the cleavage domain are in operative linkage if, in the fusion polypeptide, the Cas DNA-binding domain portion is able to bind its target site and/or its binding site, while the cleavage domain is able to cleave DNA in the vicinity of the target site.

A “functional fragment” of a protein, polypeptide or nucleic acid is a protein, polypeptide or nucleic acid whose sequence is not identical to the full-length protein, polypeptide or nucleic acid, yet retains the same function as the full-length protein, polypeptide or nucleic acid. A functional fragment can possess more, fewer, or the same number of residues as the corresponding native molecule, and/or can contain one or more amino acid or nucleotide substitutions. Methods for determining the function of a nucleic acid (e.g., coding function, ability to hybridize to another nucleic acid) are well-known in the art. Similarly, methods for determining protein function are well-known. For example, the DNA-binding function of a polypeptide can be determined, for example, by filter-binding, electrophoretic mobility-shift, or immunoprecipitation assays. DNA cleavage can be assayed by gel electrophoresis. See Ausubel et al., supra. The ability of a protein to interact with another protein can be determined, for example, by co-immunoprecipitation, two-hybrid assays or complementation, both genetic and biochemical. See, for example, Fields et al. (1989) Nature 340: 245-246; U.S. Pat. No. 5,585,245 and PCT WO 98/44350.

A “vector” is capable of transferring gene sequences to target cells. Typically, “vector construct,” “expression vector,” and “gene transfer vector,” mean any nucleic acid construct capable of directing the expression of a gene of interest and which can transfer gene sequences to target cells. Thus, the term includes cloning, and expression vehicles, as well as integrating vectors.

A “reporter gene” or “reporter sequence” refers to any sequence that produces a protein product that is easily measured, preferably although not necessarily in a routine assay. Suitable reporter genes include, but are not limited to, sequences encoding proteins that mediate antibiotic resistance (e.g., ampicillin resistance, neomycin resistance, G418 resistance, puromycin resistance), sequences encoding colored or fluorescent or luminescent proteins (e.g., green fluorescent protein, enhanced green fluorescent protein, red fluorescent protein, luciferase), and proteins which mediate enhanced cell growth and/or gene amplification (e.g., dihydrofolate reductase). Epitope tags include, for example, one or more copies of FLAG, His, myc, Tap, HA or any detectable amino acid sequence. “Expression tags” include sequences that encode reporters that may be operably linked to a desired gene sequence in order to monitor expression of the gene of interest.

### Nucleases

### A. DNA Binding Domains

The methods described herein increase the activity of nuclease. The nucleases useful in the methods described herein comprise any DNA-binding domain that specifically binds to a target sequence. Any polynucleotide or polypeptide DNA-binding domain can be used in the compositions and methods disclosed herein, for example DNA-binding proteins (e.g., ZFPs or TALEs) or DNA-binding polynucleotides (e.g., single guide RNAs).

In certain embodiments, the DNA binding domain, comprises a zinc finger protein. Selection of target sites; ZFPs and methods for design and construction of fusion proteins (and polynucleotides encoding same) are known to those of skill in the art and described in detail in U.S. Pat. Nos. 6,140,081; 5,789,538; 6,453,242; 6,534,261; 5,925,523; 6,007,988; 6,013,453; and 6,200,759; International Patent Publication Nos. WO 95/19431; WO 96/06166; WO 98/53057; WO 98/54311; WO 00/27878; WO 01/60970; WO 01/88197; WO 02/099084; WO 98/53058; WO 98/53059; WO 98/53060; WO 02/016536 and WO 03/016496.

In certain embodiments, the DNA-binding domain comprises a naturally occurring or engineered (non-naturally occurring) TAL effector (TALE) DNA binding domain. See, e.g., U.S. Pat. No. 8,586,526, incorporated by reference in its entirety herein.

The plant pathogenic bacteria of the genus Xanthomonas are known to cause many diseases in important crop plants. Pathogenicity of Xanthomonas depends on a conserved type III secretion (T3S) system which injects more than 25 different effector proteins into the plant cell. Among these injected proteins are transcription activator-like effectors (TALE) which mimic plant transcriptional activators and manipulate the plant transcriptome (see Kay et al (2007) Science 318: 648-651). These proteins contain a DNA binding domain and a transcriptional activation domain. One of the most well characterized TALEs is AvrBs3 from Xanthomonas campestgris pv. Vesicatoria (see Bonas et al (1989) Mol Gen Genet 218: 127-136 and WO2010079430). TALEs contain a centralized domain of tandem repeats, each repeat containing approximately 34 amino acids, which are key to the DNA binding specificity of these proteins. In addition, they contain a nuclear localization sequence and an acidic transcriptional activation domain (for a review see Schornack S, et al (2006) J Plant Physiol 163(3): 256-272). In addition, in the phytopathogenic bacteria Ralstonia solanacearum two genes, designated brg11 and hpx17 have been found that are homologous to the AvrBs3 family of Xanthomonas in the R. solanacearum biovar 1 strain GMI1000 and in the biovar 4 strain RS1000 (See Heuer et al (2007) Appl and Envir Micro 73(13): 4379-4384). These genes are 98.9% identical in nucleotide sequence to each other but differ by a deletion of 1,575 bp in the repeat domain of hpx17. However, both gene products have less than 40% sequence identity with AvrBs3 family proteins of Xanthomonas.

Specificity of these TALEs depends on the sequences found in the tandem repeats. The repeated sequence comprises approximately 102 bp and the repeats are typically 91-100% homologous with each other (Bonas et al, ibiid). Polymorphism of the repeats is usually located at positions 12 and 13 and there appears to be a one-to-one correspondence between the identity of the hypervariable diresidues at positions 12 and 13 with the identity of the contiguous nucleotides in the TALE's target sequence (see Moscou and Bogdanove (2009) Science 326: 1501 and Boch et al (2009) Science 326: 1509-1512). Experimentally, the code for DNA recognition of these TALEs has been determined such that an HD sequence at positions 12 and 13 leads to a binding to cytosine (C), NG binds to T, NI to A, C, G or T, NN binds to A or G, and NG binds to T. These DNA binding repeats have been assembled into proteins with new combinations and numbers of repeats, to make artificial transcription factors that are able to interact with new sequences. In addition, U.S. Pat. No. 8,586,526 and U.S. Publication No. 20130196373, incorporated by reference in their entireties herein, describe TALEs with N-cap polypeptides, C-cap polypeptides (e.g., +63, +231 or +278) and/or novel (atypical) RVDs.

In still further embodiments, the DNA-binding domain comprises a single-guide RNA of a CRISPR/Cas system, for example sgRNAs as disclosed in 20150056705. Compelling evidence has recently emerged for the existence of an RNA-mediated genome defense pathway in archaea and many bacteria that has been hypothesized to parallel the eukaryotic RNAi pathway (for reviews, see Godde and Bickerton, 2006. J. Mol. Evol. 62: 718-729; Lillestol et al., 2006. Archaea 2: 59-72; Makarova et al., 2006. Biol. Direct 1: 7.; Sorek et al., 2008. Nat. Rev. Microbiol. 6: 181-186). Known as the CRISPR-Cas system or prokaryotic RNAi (pRNAi), the pathway is proposed to arise from two evolutionarily and often physically linked gene loci: the CRISPR (clustered regularly interspaced short palindromic repeats) locus, which encodes RNA components of the system, and the cas (CRISPR-associated) locus, which encodes proteins (Jansen et al., 2002. Mol. Microbiol. 43: 1565-1575; Makarova et al., 2002. Nucleic Acids Res. 30: 482-496; Makarova et al., 2006. Biol. Direct 1: 7; Haft et al., 2005. PLoS Comput. Biol. 1: e60). CRISPR loci in microbial hosts contain a combination of CRISPR-associated (Cas) genes as well as non-coding RNA elements capable of programming the specificity of the CRISPR-mediated nucleic acid cleavage. The individual Cas proteins do not share significant sequence similarity with protein components of the eukaryotic RNAi machinery, but have analogous predicted functions (e.g., RNA binding, nuclease, helicase, etc.) (Makarova et al., 2006. Biol. Direct 1: 7). The CRISPR-associated (cas) genes are often associated with CRISPR repeat-spacer arrays. More than forty different Cas protein families have been described. Of these protein families, Cas1 appears to be ubiquitous among different CRISPR/Cas systems. Particular combinations of cas genes and repeat structures have been used to define 8 CRISPR subtypes (Ecoli, Ypest, Nmeni,

Dvulg, Tneap, Hmari, Apern, and Mtube), some of which are associated with an additional gene module encoding repeat-associated mysterious proteins (RAMPs). More than one CRISPR subtype may occur in a single genome. The sporadic distribution of the CRISPR/Cas subtypes suggests that the system is subject to horizontal gene transfer during microbial evolution.

The Type II CRISPR, initially described in S. pyogenes, is one of the most well characterized systems and carries out targeted DNA double-strand break in four sequential steps. First, two non-coding RNA, the pre-crRNA array and tracrRNA, are transcribed from the CRISPR locus. Second, tracrRNA hybridizes to the repeat regions of the pre-crRNA and mediates the processing of pre-crRNA into mature crRNAs containing individual spacer sequences where processing occurs by a double strand-specific RNase III in the presence of the Cas9 protein. Third, the mature crRNA:tracrRNA complex directs Cas9 to the target DNA via Watson-Crick base-pairing between the spacer on the crRNA and the protospacer on the target DNA next to the protospacer adjacent motif (PAM), an additional requirement for target recognition. In addition, the tracrRNA must also be present as it base pairs with the crRNA at its 3′ end, and this association triggers Cas9 activity. Finally, Cas9 mediates cleavage of target DNA to create a double-stranded break within the protospacer. In addition, CRISPR/Cas system Cpf1 has recently been described from Acidominococcus and Lachnospiraceae (Zetsche et al (2015) Cell 163: 1-16). Thus, in some embodiments, the CRISPR-Cpf1 system is used. The CRISPR-Cpf1 system, identified in Francisella spp, is a class 2 CRISPR-Cas system that mediates robust DNA interference in human cells. Although functionally conserved, Cpf1 and Cas9 differ in many aspects including in their guide RNAs and substrate specificity (see Fagerlund et al, (2015) Genom Bio 16: 251). A major difference between Cas9 and Cpf1 proteins is that Cpf1 does not utilize tracrRNA, and thus requires only a crRNA. The FnCpf1 crRNAs are 42-44 nucleotides long (19-nucleotide repeat and 23-25-nucleotide spacer) and contain a single stem-loop, which tolerates sequence changes that retain secondary structure. In addition, the Cpf1 crRNAs are significantly shorter than the ˜100-nucleotide engineered sgRNAs required by Cas9, and the PAM requirements for FnCpf1 are 5′-TTN-3′ and 5′-CTA-3′ on the displaced strand. Although both Cas9 and Cpf1 make double strand breaks in the target DNA, Cas9 uses its RuvC- and HNH-like domains to make blunt-ended cuts within the seed sequence of the guide RNA, whereas Cpf1 uses a RuvC-like domain to produce staggered cuts outside of the seed. Because Cpf1 makes staggered cuts away from the critical seed region, NHEJ will not disrupt the target site, therefore ensuring that Cpf1 can continue to cut the same site until the desired HDR recombination event has taken place. Thus, in the methods and compositions described herein, it is understood that the term “Cas” includes both Cas9 and Cpf1 proteins. Thus, as used herein, a “CRISPR/Cas system” refers both CRISPR/Cas and/or CRISPR/Cpf1 systems, including both nuclease and/or transcription factor systems. Activity of the CRISPR/Cas system comprises of three steps: (i) insertion of alien DNA sequences into the CRISPR array to prevent future attacks, in a process called ‘adaptation,’ (ii) expression of the relevant proteins, as well as expression and processing of the array, followed by (iii) RNA-mediated interference with the alien nucleic acid. Thus, in the bacterial cell, several of the so-called ‘Cas’ proteins are involved with the natural function of the CRISPR/Cas system.

Type II CRISPR systems have been found in many different bacteria. BLAST searches on publically available genomes by Fonfara et al ((2013) Nuc Acid Res 42(4): 2377-2590) found Cas9 orthologs in 347 species of bacteria. Additionally, this group demonstrated in vitro CRISPR/Cas cleavage of a DNA target using Cas9 orthologs from S. pyogenes, S. mutans, S. therophilus, C. jejuni, N. meningitides, P. multocida and F. novicida. Thus, the term “Cas9” refers to an RNA guided DNA nuclease comprising a DNA binding domain and two nuclease domains, where the gene encoding the Cas9 may be derived from any suitable bacteria.

The Cas9 protein has at least two nuclease domains: one nuclease domain is similar to a HNH endonuclease, while the other resembles a Ruv endonuclease domain. The HNH-type domain appears to be responsible for cleaving the DNA strand that is complementary to the crRNA while the Ruv domain cleaves the non-complementary strand. The Cas 9 nuclease can be engineered such that only one of the nuclease domains is functional, creating a Cas nickase (see Jinek et al, ibid). Nickases can be generated by specific mutation of amino acids in the catalytic domain of the enzyme, or by truncation of part or all of the domain such that it is no longer functional. Since Cas 9 comprises two nuclease domains, this approach may be taken on either domain. A double strand break can be achieved in the target DNA by the use of two such Cas 9 nickases. The nickases will each cleave one strand of the DNA and the use of two will create a double strand break.

The requirement of the crRNA-tracrRNA complex can be avoided by use of an engineered “single-guide RNA” (sgRNA) that comprises the hairpin normally formed by the annealing of the crRNA and the tracrRNA (see Jinek et al (2012) Science 337: 816 and Cong et al (2013) Sciencexpress/10.1126/science.1231143). In S. pyrogenes, the engineered tracrRNA:crRNA fusion, or the sgRNA, guides Cas9 to cleave the target DNA when a double strand RNA:DNA heterodimer forms between the Cas associated RNAs and the target DNA. This system comprising the Cas9 protein and an engineered sgRNA containing a PAM sequence has been used for RNA guided genome editing (see Ramalingam, ibid) and has been useful for zebrafish embryo genomic editing in vivo (see Hwang et al (2013) Nature Biotechnology 31 (3):227) with editing efficiencies similar to ZFNs and TALENs.

The primary products of the CRISPR loci appear to be short RNAs that contain the invader targeting sequences, and are termed guide RNAs or prokaryotic silencing RNAs (psiRNAs) based on their hypothesized role in the pathway (Makarova et al., 2006. Biol. Direct 1: 7; Hale et al., 2008. RNA, 14: 2572-2579). RNA analysis indicates that CRISPR locus transcripts are cleaved within the repeat sequences to release ˜60- to 70-nt RNA intermediates that contain individual invader targeting sequences and flanking repeat fragments (Tang et al. 2002. Proc. Natl. Acad. Sci. 99: 7536-7541; Tang et al., 2005. Mol. Microbiol. 55: 469-481; Lillestol et al. 2006. Archaea 2: 59-72; Brouns et al. 2008. Science 321: 960-964; Hale et al, 2008. RNA, 14: 2572-2579). In the archaeon Pyrococcus furiosus, these intermediate RNAs are further processed to abundant, stable ˜35- to 45-nt mature psiRNAs (Hale et al. 2008. RNA, 14: 2572-2579).

The requirement of the crRNA-tracrRNA complex can be avoided by use of an engineered “single-guide RNA” (sgRNA) that comprises the hairpin normally formed by the annealing of the crRNA and the tracrRNA (see Jinek et al (2012) Science 337: 816 and Cong et al (2013) Sciencexpress/10.1126/science.1231143). In S. pyrogenes, the engineered tracrRNA:crRNA fusion, or the sgRNA, guides Cas9 to cleave the target DNA when a double strand RNA:DNA heterodimer forms between the Cas associated RNAs and the target DNA. This system comprising the Cas9 protein and an engineered sgRNA containing a PAM sequence has been used for RNA guided genome editing (see Ramalingam ibid) and has been useful for zebrafish embryo genomic editing in vivo (see Hwang et al (2013) Nature Biotechnology 31 (3): 227) with editing efficiencies similar to ZFNs and TALENs.

Chimeric or sgRNAs can be engineered to comprise a sequence complementary to any desired target. In some embodiments, a guide sequence is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, a guide sequence is less than about 75, 50, 45, 40, 35, 30, 25, 20, 15, 12, or fewer nucleotides in length. In some embodiments, the RNAs comprise 22 bases of complementarity to a target and of the form G[n19], followed by a protospacer-adjacent motif (PAM) of the form NGG or NAG for use with a S. pyogenes CRISPR/Cas system. Thus, in one method, sgRNAs can be designed by utilization of a known ZFN target in a gene of interest by (i) aligning the recognition sequence of the ZFN heterodimer with the reference sequence of the relevant genome (human, mouse, or of a particular plant species); (ii) identifying the spacer region between the ZFN half-sites; (iii) identifying the location of the motif G[N20]GG that is closest to the spacer region (when more than one such motif overlaps the spacer, the motif that is centered relative to the spacer is chosen); (iv) using that motif as the core of the sgRNA. This method advantageously relies on proven nuclease targets. Alternatively, sgRNAs can be designed to target any region of interest simply by identifying a suitable target sequence the conforms to the G[n20]GG formula. Along with the complementarity region, an sgRNA may comprise additional nucleotides to extend to tail region of the tracrRNA portion of the sgRNA (see Hsu et al (2013) Nature Biotech doi: 10.1038/nbt.2647). Tails may be of +67 to +85 nucleotides, or any number therebetween with a preferred length of +85 nucleotides. Truncated sgRNAs may also be used, “tru-gRNAs” (see Fu et al, (2014) Nature Biotech 32(3): 279). In tru-gRNAs, the complementarity region is diminished to 17 or 18 nucleotides in length.

Further, alternative PAM sequences may also be utilized, where a PAM sequence can be NAG as an alternative to NGG (Hsu 2014, ibid) using a S. pyogenes Cas9. Additional PAM sequences may also include those lacking the initial G (Sander and Joung (2014) Nature Biotech 32(4): 347). In addition to the S. pyogenes encoded Cas9 PAM sequences, other PAM sequences can be used that are specific for Cas9 proteins from other bacterial sources. For example, the PAM sequences shown below (adapted from Sander and Joung, ibid, and Esvelt et al, (2013) Nat Meth 10(11): 1116) are specific for these Cas9 proteins:

| Species | PAM |
|---|---|
| S. pyogenes | NGG |
| S. pyogenes | NAG |
| S. mutans | NGG |
| S. thermophilius | NGGNG |
| S. thermophilius | NNAAAW |
| S. thermophilius | NNAGAA |
| S. thermophilius | NNNGATT |
| C. jejuni | NNNNACA |
| N. meningitides | NNNNGATT |
| P. multocida | GNNNCNNA |
| F. novicida | NG |
| Acidominococcus and Lachnospiraceae | TTN |

Thus, a suitable target sequence for use with a S. pyogenes CRISPR/Cas system can be chosen according to the following guideline: [n17, n18, n19, or n20](G/A)G. Alternatively the PAM sequence can follow the guideline G[n17, n18, n19, n20](G/A)G. For Cas9 proteins derived from non-S. pyogenes bacteria, the same guidelines may be used where the alternate PAMs are substituted in for the S. pyogenes PAM sequences.

Most preferred is to choose a target sequence with the highest likelihood of specificity that avoids potential off target sequences. These undesired off target sequences can be identified by considering the following attributes: i) similarity in the target sequence that is followed by a PAM sequence known to function with the Cas9 protein being utilized; ii) a similar target sequence with fewer than three mismatches from the desired target sequence; iii) a similar target sequence as in ii), where the mismatches are all located in the PAM distal region rather than the PAM proximal region (there is some evidence that nucleotides 1-5 immediately adjacent or proximal to the PAM, sometimes referred to as the ‘seed’ region (Wu et al (2014) Nature Biotech doi:10.1038/nbt2889) are the most critical for recognition, so putative off target sites with mismatches located in the seed region may be the least likely be recognized by the sg RNA); and iv) a similar target sequence where the mismatches are not consecutively spaced or are spaced greater than four nucleotides apart (Hsu 2014, ibid). Thus, by performing an analysis of the number of potential off target sites in a genome for whichever CRIPSR/Cas system is being employed, using these criteria above, a suitable target sequence for the sgRNA may be identified.

In certain embodiments, Cas protein may be a “functional derivative” of a naturally occurring Cas protein. A “functional derivative” of a native sequence polypeptide is a compound having a qualitative biological property in common with a native sequence polypeptide. “Functional derivatives” include, but are not limited to, fragments of a native sequence and derivatives of a native sequence polypeptide and its fragments, provided that they have a biological activity in common with a corresponding native sequence polypeptide. A biological activity contemplated herein is the ability of the functional derivative to hydrolyze a DNA substrate into fragments. The term “derivative” encompasses both amino acid sequence variants of polypeptide, covalent modifications, and fusions thereof. In some aspects, a functional derivative may comprise a single biological property of a naturally occurring Cas protein. In other aspects, a function derivative may comprise a subset of biological properties of a naturally occurring Cas protein. Suitable derivatives of a Cas polypeptide or a fragment thereof include but are not limited to mutants, fusions, covalent modifications of Cas protein or a fragment thereof. Cas protein, which includes Cas protein or a fragment thereof, as well as derivatives of Cas protein or a fragment thereof, may be obtainable from a cell or synthesized chemically or by a combination of these two procedures. The cell may be a cell that naturally produces Cas protein, or a cell that naturally produces Cas protein and is genetically engineered to produce the endogenous Cas protein at a higher expression level or to produce a Cas protein from an exogenously introduced nucleic acid, which nucleic acid encodes a Cas that is same or different from the endogenous Cas. In some case, the cell does not naturally produce Cas protein and is genetically engineered to produce a Cas protein.

Exemplary CRISPR/Cas nuclease systems targeted to specific genes are disclosed for example, in U.S. Publication No. 20150056705.

### B. Cleavage Domains

The nucleases used in methods described herein also include a cleavage (nuclease) domain. The nuclease domain may be derived from any nuclease, for example any endonuclease or exonuclease. Non-limiting examples of suitable nuclease (cleavage) domains that may be used with DNA-binding domains as described herein include domains from any restriction enzyme, for example a Type IIS Restriction Enzyme (e.g., FokI). In certain embodiments, the cleavage domains are cleavage half-domains that require dimerization for cleavage activity. See, e.g., U.S. Patent Nos. 8,586,526; 8,409,861 and 7,888,121, incorporated by reference in their entireties herein. In general, two fusion proteins are required for cleavage if the fusion proteins comprise cleavage half-domains. Alternatively, a single protein comprising two cleavage half-domains can be used. The two cleavage half-domains can be derived from the same endonuclease (or functional fragments thereof), or each cleavage half-domain can be derived from a different endonuclease (or functional fragments thereof). In addition, the target sites for the two fusion proteins are preferably disposed, with respect to each other, such that binding of the two fusion proteins to their respective target sites places the cleavage half-domains in a spatial orientation to each other that allows the cleavage half-domains to form a functional cleavage domain, e.g., by dimerizing.

The nuclease domain may also be derived any meganuclease (homing endonuclease) domain with cleavage activity may also be used with the nucleases described herein, including but not limited to I-SceI, I-CeuI, PI-PspI, PI-Sce, I-SceIV, I-CsmI, I-PanI, I-SceII, I-PpoI, I-SceIII, I-CreI, I-TevI, I-TevII and I-TevIII.

In addition, cleavage domains may include one or more alterations as compared to wild-type, for example for the formation of obligate heterodimers that reduce or eliminate off-target cleavage effects. See, e.g., U.S. Pat. Nos. 7,914,796; 8,034,598; and 8,623,618, incorporated by reference in their entireties herein.

Nucleases as described herein may generate double- or single-stranded breaks in a double-stranded target (e.g., gene). The generation of single-stranded breaks (“nicks”) is described, for example in U.S. Pat. Nos. 9,200,266 and 8,703,489, incorporated herein by reference which describes how mutation of the catalytic domain of one of the nucleases domains results in a nickase.

### Cells

Any host cell wherein a genomic modification is desired may be used in the practice of the present disclosure. Prokaryotic (e.g., bacterial) or eukaryotic (e.g., yeast, plant, fungal, piscine and mammalian cells such as feline, canine, murine, bovine, porcine and human) cells can be used, with eukaryotic cells being preferred. The cell types can be cell lines or natural (e.g., isolated) cells such as, for example, primary cells.

Cell lines are available, for example from the American Type Culture Collection (ATCC), or can be generated by methods known in the art, as described for example in Freshney et al., Culture of Animal Cells, A Manual of Basic Technique, 3rd ed., 1994, and references cited therein. Similarly, cells can be isolated by methods known in the art. Other non-limiting examples of cell types include cells that have or are subject to pathologies, such as cancerous cells and transformed cells, pathogenically infected cells, stem cells, fully differentiated cells, partially differentiated cells, immortalized cells and the like.

In certain embodiments, the cells are stem cells, for example CD34+ hematopoietic stem cells, including CD34+ cells derived from bone marrow (BM-derived CD34+ cells) or induced pluripotent stem cells (iPSCs).

Suitable mammalian cell lines include K562 cells, CHO (Chinese hamster ovary) cells, 293 cells, HEP-G2 cells, BaF-3 cells, Schneider cells, COS cells (monkey kidney cells expressing SV40 T-antigen), CV-1 cells, HuTu80 cells, NTERA2 cells, NB4 cells, HL-60 cells and HeLa cells, 293 cells (see, e.g., Graham et al. (1977) J. Gen. Virol. 36: 59), and myeloma cells like SP2 or NS0 (see, e.g., Galfre and Milstein (1981)Meth. Enzymol. 73(B): 3 46), rat C6 cells, and porcine Pk15 cells. Peripheral blood mononucleocytes (PBMCs) or T-cells can also serve as hosts. Other eukaryotic cells include, for example, insect (e.g., sp. frugiperda), fungal cells, including yeast (e.g., S. cerevisiae, S. pombe, P. pastoris, K lactis, H. polymorpha), and plant cells (Fleer, R. (1992) Current Opinion in Biotechnology 3: 486 496).

### Cold Shock Conditions

The methods described herein involve subjecting the cells, RNAs (e.g. nucleases (in polynucleotide form), mRNAs encoding other polypeptides, RNAis, miRNAs) and/or optional donor sequences to a period of extreme (severe) cold shock (below 15° C.) before, during and/or after introduction of the nuclease(s) and/or donor polynucleotide. Typically, the cells, RNAs and optional donor sequences are cold-shocked before and during introduction of (e.g., transfection with) the RNA and/or donor nucleotide into the cell.

The period of time of extreme cold shock can vary from minutes to hours.

In certain embodiments, the cells are cold-shocked for between 1 and 60 minutes (or any time therebetween) before, during and/or after introduction of the nucleases (and optional donors) into the cells. In certain embodiments, the RNAs, cells and donors subject to cold shock for 0-60 minutes before introduction of the RNAs and/or donors into the cells as well as during introduction (e.g., transfection) and for 0-60 minutes (or any time therebetween) after introduction of the RNAs and/or donors (e.g., the cell-RNA mixture is subject to cold-shock for 0-60 minutes). It will be apparent that the period of cold shock will also vary depending on the cell type into which the RNA is introduced. In certain embodiments, all components (cells, RNAs and/or optional donors) are cold-shocked for 1, 2, 3, 4, 5, 6, 7, 8, 9, 10 minutes before introduction of the RNAs and/or donors, during introduction and for 1, 2, 3, 4, 5, 6, 7, 8, 9, 10 or more minutes the RNAs are introduced (i.e., the cell-RNA mixture is cold-shocked). In certain embodiments, more than one species of RNA is introduced.

Likewise, the temperature of extreme cold-shock is any temperature below about 15° C., including but not limited, 15° C., 14° C., 13° C., 12° C., 11° C., 10° C., 9° C., 8° C., 7° C., 6° C., 5° C., 4° C., 3° C., 2° C., 1° C., 0° C., −1° C., −2° C., −3° C., −4° C., −5° C., −6° C., −7° C., −8° C., −9° C., −10° C., −11° C., −12° C., −13° C., −14° C., −15° C. or even lower (or any value therebetween). The severe cold shock may be achieved by placing the cells, RNAs and/or donors on ice or in the refrigerator. Furthermore, the temperature can vary during the period of cold-shocking, so long as it remains under about 15° C.

The methods as described herein may further comprise culturing the cells subjected to extreme cold-shock at a temperature below their optimal growth temperature for a period of time. See, e.g., U.S. Pat. No. 8,772,008. In certain embodiments, following the extreme cold-shock, the cells expressing a nuclease are cultured at between about 27° C. and about 33° C. for a period of hours (e.g., 1-24 hours) to 4 days.

### Delivery

The nucleases, polynucleotides encoding these nucleases, donor polynucleotides and compositions comprising the proteins and/or polynucleotides described herein may be delivered to the cells by any suitable means. See, e.g., U.S. Pat. Nos. 6,453,242; 6,503,717; 6,534,261; 6,599,692; 6,607,882; 6,689,558; 6,824,978; 6,933,113; 6,979,539; 7,013,219; and 7,163,824, the disclosures of all of which are incorporated by reference herein in their entireties.

Nucleases and/or donor constructs as described herein may be delivered in polynucleotide form using any vector system, including but not limited to plasmid vectors, retroviral vectors, lentiviral vectors, adenovirus vectors, poxvirus vectors; herpesvirus vectors and adeno-associated virus vectors, etc. In certain embodiments, the nucleases and/or donor constructs are delivered in mRNA form. Furthermore, it will be apparent that any of these delivery systems may comprise one or more of the sequences involved in genomic modification. Thus, when one or more nucleases and/or a donor are introduced into the cell, the nucleases and/or donor polynucleotide may be delivered in the same form (e.g., mRNA). Alternatively, the nucleases and donors may be delivered in different forms (e.g., mRNA nuclease, viral or non-viral vector for donor).

Methods of delivery of non-viral nucleic acids include electroporation, lipofection, microinjection, biolistics, virosomes, liposomes, immunoliposomes, polycation or lipid:nucleic acid conjugates, naked DNA, artificial virions, and agent-enhanced uptake of DNA. Sonoporation using, e.g., the Sonitron 2000 system (Rich-Mar) can also be used for delivery of nucleic acids.

Additional exemplary nucleic acid delivery systems suitable for introducing RNA into the cells described herein include those provided by Amaxa Biosystems (Cologne, Germany), Maxcyte, Inc. (Rockville, Md.), BTX Molecular Delivery Systems (Holliston, Mass.) and Copernicus Therapeutics Inc., (see for example U.S. Pat. No. 6,008,336). Lipofection is described in e.g., U.S. Pat. Nos. 5,049,386; 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Felgner, WO 91/17424, WO 91/16024.

Still further suitable nucleic acid delivery systems for introducing polynucleotides (e.g., RNAs) into the cells described herein include microfluidic devices such as those described in US Patent Publication 2011/0213288, W02013/059343. Certain microfluidic devices are commercially available as well (SQZ Biotech, Boston and Somerville, Mass.).

### Kits

Also provided are kits for performing any of the above methods. The kits typically contain nucleases in polynucleotide form, polypeptide encoding mRNAs,

RNAis, miRNAs and/or donor polynucleotides as described herein as well as instructions for cold-shocking these components. The kits can also contain cells, buffers for transformation of cells, culture media for cells, and/or buffers for performing assays. Typically, the kits also contain a label which includes any material such as instructions, packaging or advertising leaflet that is attached to or otherwise accompanies the other components of the kit.

The following Examples relate to exemplary embodiments of the present disclosure in which the nuclease comprises a zinc finger nuclease (ZFN) or CRISPR/Cas nuclease system. It will be appreciated that this is for purposes of exemplification only and that other nucleases can be used, for instance homing endonucleases (meganucleases) with engineered DNA-binding domains and/or fusions of naturally occurring of engineered homing endonucleases (meganucleases) DNA-binding domains and heterologous cleavage domains or TAL-effector domain nuclease fusion proteins. In addition, it will be appreciated that these Examples serve to exemplify methods that can be used with other RNAs, for example mRNAs encoding other polypeptides, RNAis and miRNAs. cl EXAMPLES

### Example 1: Transfection Efficiency After Cold-Shock Using GFP mRNA

Bone marrow (BM) CD34+ cells were isolated from human bone marrow aspirates by first depleting erythrocytes, and then enriching CD34+ hematopoietic stem and progenitor cells (HSPC) using paramagnetic nanobead coupled CD34+ antibody with CliniMACS Prodigy (Miltenyi).

Bone marrow-derived CD34+ cells (BM-CD34+ ) were washed none, 1 or 3 times with Maxcyte EP buffer with 10% X vivo 10 medium and transfected with GFP mRNA as follows. BM-CD34+ cells were cultured for at least 1 day after isolation, or thaw from cryo-preserved cells in X vivo 10 media with cytokines. The appropriate number of cells was collected and centrifuged for 5-10 minutes at 100-250× g. The supernatant was aspirated and cells optionally washed 1, 2, 3 or 4 times with MaxCyte EP buffer with 0.1% human serum albumin (>10× cell pellet volume). Following the final washing, cells were re-suspended in EP buffer with no albumin to concentrations about 3×10⁷-2×10⁸ cells/ml (using the cell count as determined previously). The cells, GFP mRNA and Processing Assembly (PA) were placed on ice. The mRNA was added to the cell suspension to a desired final concentration (50 μg to 300 μg/ml). The cells and mRNA mixture were then transferred to PA, and electroporated by MaxCyte apparatus using the optimized program. Cells were then removed from the PA and transferred to a well of a tissue culture plate (proportionally about 80 μL cells/cm² bottom surface or about 80 μL cell/well, 48 well flat-bottom plate) and the plates incubated in a 37° C. incubator for approximately 20 minutes. Subsequently, cells were plated in resuspended in pre-warmed media and cultured at 30° C. or 37° C. until analysis by FACS for GFP expression.

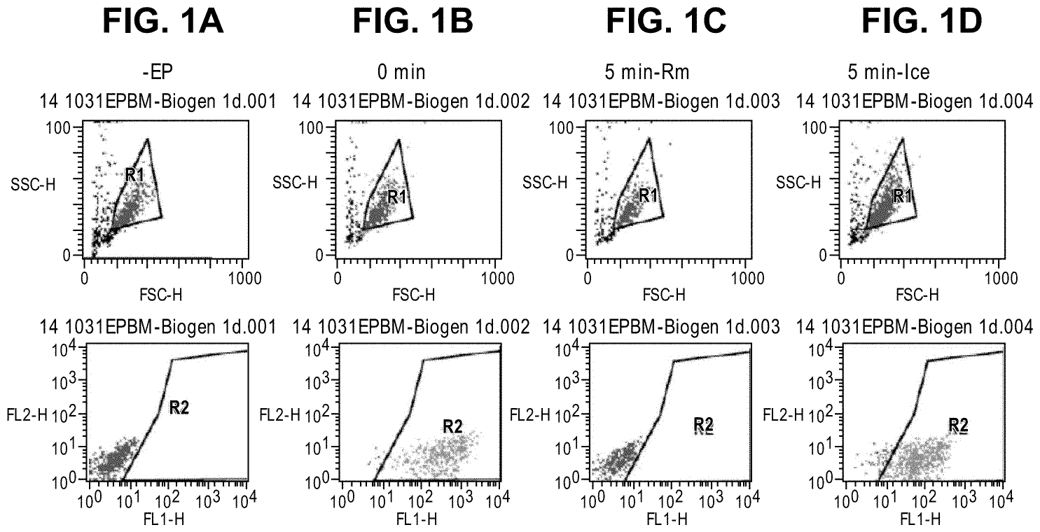

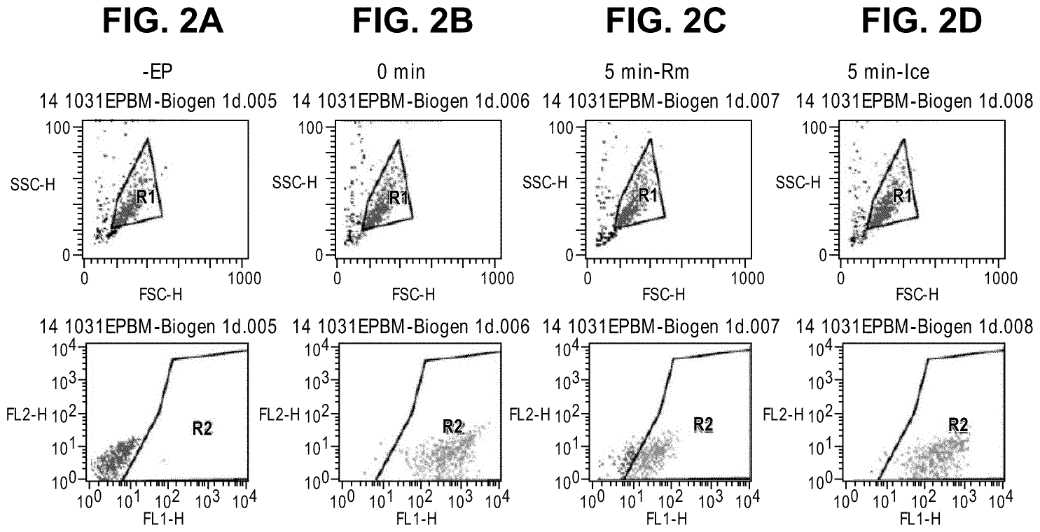

As shown in FIGS. 1 and 2, holding the cell-mRNA mixture on ice (cold shock) dramatically improved transfection efficiency (as determined by GFP-expressing cells) as compared to cells held at room temperature (each for 5 minutes). In addition, as shown in FIG. 2, cells washed 3 times in EP buffer resulted in further increases in transfection efficiencies.

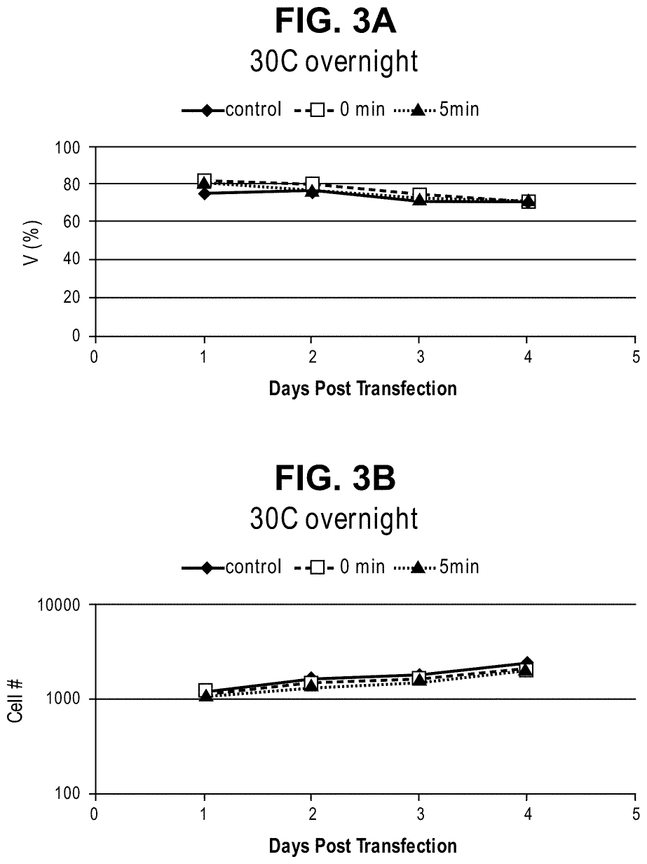

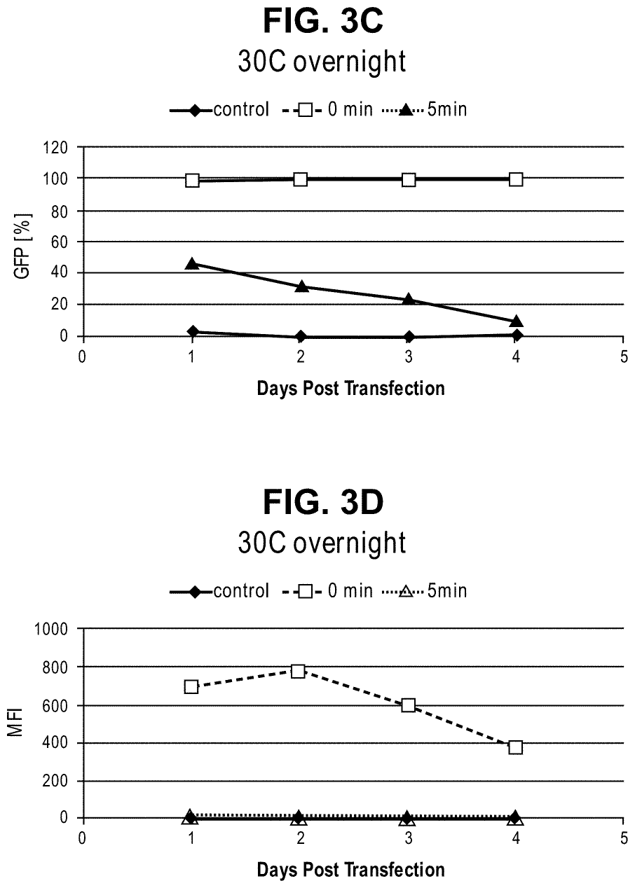

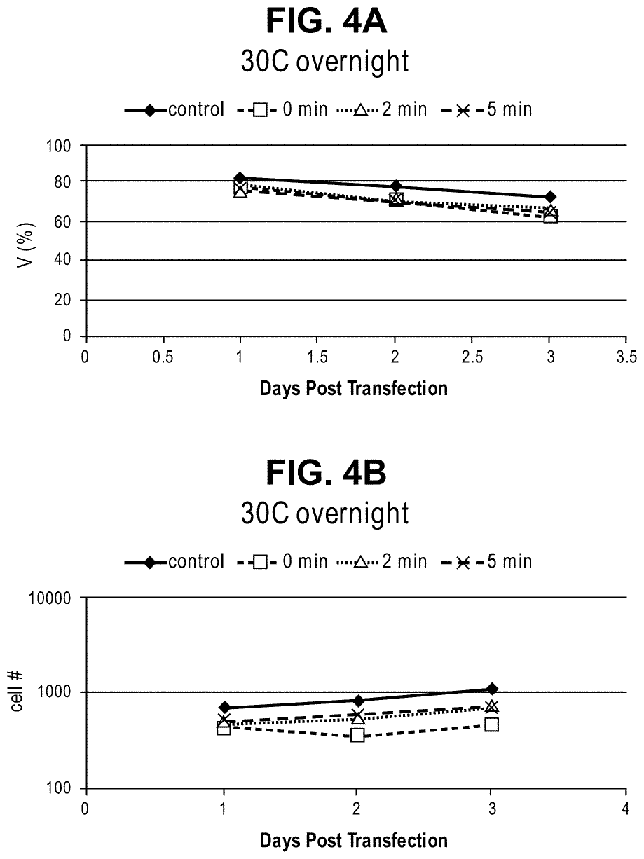

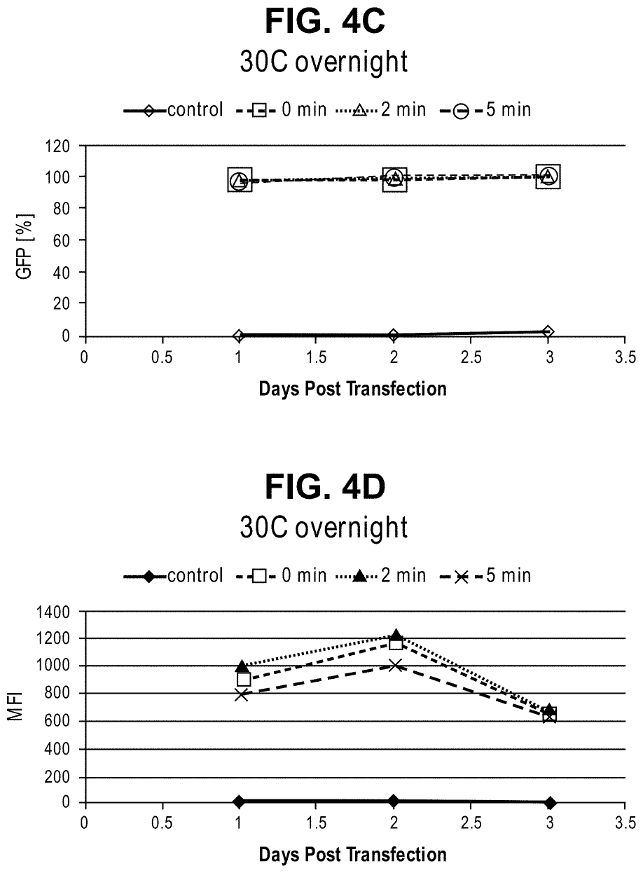

As shown in FIG. 3, despite all groups showing the same viability (FIG. 3A) and cell number (FIG. 3B), mixing of cells and mRNA at room temperature, and followed immediately by electroporation showed significantly increased GFP expression as compared to cells that were held at room temperature for 5 minutes after mixing with mRNA prior to electroporation (FIGS. 3C and 3D). By contrast, as shown in FIG. 4, cells that previously chilled, mixed with ice-cold mRNA, and held on ice for 0, 2 or 5 minutes prior to electroporation, all showed high levels of GFP expression (FIGS. 4C and 4D) with good viability and survival (FIG. 4A and 4B).

These results demonstrate that pre-chilled cells on ice, and mixing with cold mRNA prior to transfection exhibit greatly increased transfection efficiencies as compared to the cells and cell-mRNA mix at room temperature before transfection.

### Example 2: Increased Gene Editing in BM-CD34+ Using CRISPR/Cas

Nuclease-mediated genomic editing in BM-CD34+ cells was also evaluated under the cold shock conditions described herein. In particular, 200 μg/mL CRISPR/Cas nucleases (Cas 9) targeted to AAVS1 (see, e.g., U.S. Patent Publication No. 20150056705) were introduced into BM-CD34+ cells in mRNA form essentially as described in Example 1 except without 30° C. culture. Briefly, the cells were washed 0 to 4 times in EP buffer and transfected with the appropriate AAVS1-targeted CRISPR/Cas nucleases and optionally a 6 nucleotide oligo donor comprising a HindIII site as described above. To determine nuclease-mediated gene editing in cells, CEL-I mismatch assays were performed essentially as per the manufacturer's instructions (Trangenomic SURVEYOR™). To determine nuclease-mediated gene integration in cells including the HindIII donor, PCR analysis was performed to identify cells with the integrated HindIII site.

The following specific protocol was used with the MaxCyte electroporator (MaxCyte GT) according using the instructions and reagents available from the manufacturer (MaxCyte Inc, Gaithersburg, Md/).

1. Prepare the washing buffer by adding final concentration of 0.1% human serum albumin (HSA) into MaxCyte EP buffer, keep the buffer on ice.

2. Cool down the Processing Chamber (i.e. OC-400, MaxCyte) in ice without opening.

3. Culture the bone marrow CD34+ hematopoietic stem and progenitor cells (BM-CD34+ HSPC) in X-VIVO media containing glutamax and 3 cytokine cocktail [(Recombinant Human Stem Cell Factor (SCF), Recombinant Human Thrombopoeitin (TPO), and Recombinant Human Flt-3 Ligand (Flt-3L)].

5. Collect HSPC at 200 g for 7.5 min (4° C. centrifuge), aspirate the supernatant

6. Add ice cold washing buffer prepared at step 1 (100× volume of the cell pellet). Spin HSPC at 200 g for 7.5 min, remove the supernatant. Spin the cells again at 200 g for 1 min, and remove the residual supernatant.

8. Add MaxCyte EP buffer to make cell concentration around 6.25 e7/ml. Keep the cell suspension on ice.

9. Add ice-cooled mRNA (formulated in nuclease-free water) to a desired final concentration (Cas9 mRNA at ˜230 μg/ml, guided RNA at ˜200 μg/ml, or ZFN mRNA at 300 μg/ml). The added volume of total mRNA should be <20% of the total volume.

10. Transfer the cell-mRNA mixture into ice-cooled OC-400 prepared at step 2. (perform the process as quickly as possible after mRNA is added to cells).

11. Slide OC-400 into MaxCyte GT system and run the electroporation program. Remove cells from OC-400 and transfer the cells to a well (2×10\hat{ }6 cells/mL).

12. Incubate cells in a 37° C. incubator for 20 min.

13. Collect the incubated cells and resuspend the cells into pre-warmed full medium according to step 3.

The ice-chilling step was also implemented with another electroporation device (BTX, Amaxia nucleofector) with similar results using the instructions and other materials provided by the manufacturer.

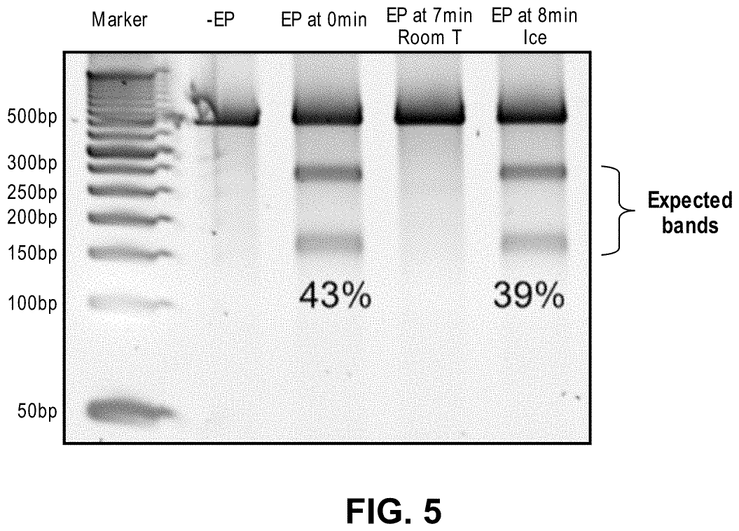

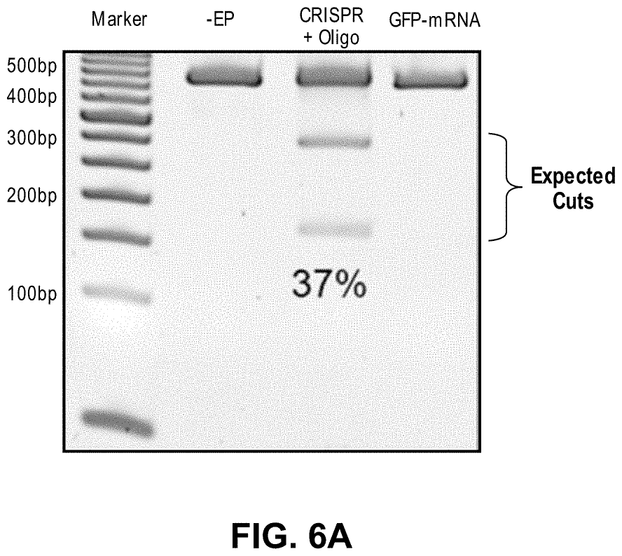

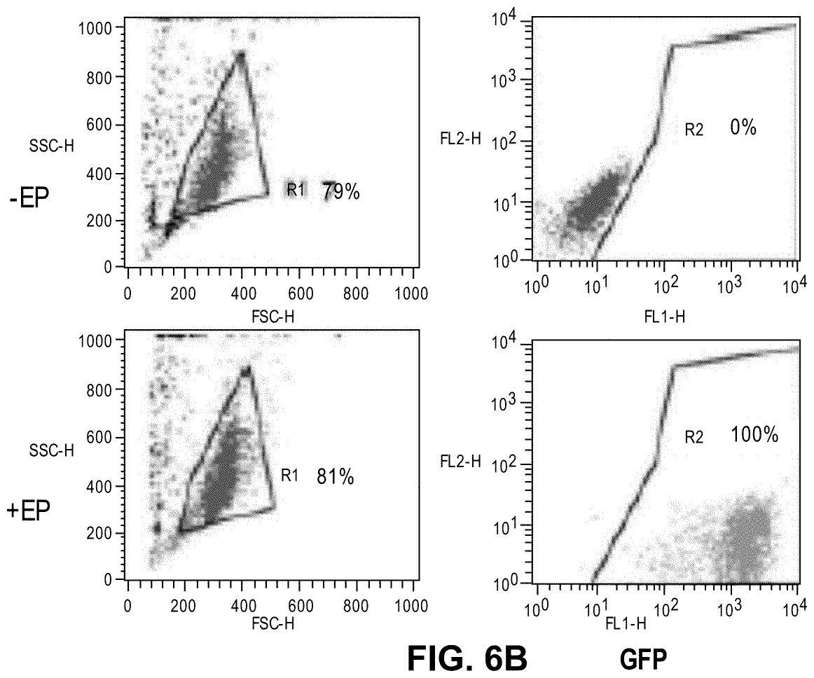

As shown in FIG. 5, high efficient gene editing was observed when cells were electroporated immediately after mixing with mRNA (EP at 0min), Incubating the cell-mRNA mix at room temperature for 7 min (EP at 7 min Room T) prior to electroporation resulted in no detectable gene editing. Chilled the cells and incubating cells-mRNA mix on ice for 8 min (EP at 8min Ice) retained the high gene editing activity. Similarly, as shown in FIG. 6A, integration of the HindIII oligo was seen in 37% of cold-shock cells treated with the CRISPR/Cas nuclease and the donor while no integration was seen in the control cells. Under the ice-chilling conditions, the electroporation efficiency of mRNA can be as high as close to 100%, as indicated in

FIG. 6B, when GFP mRNA was used as a reporter.

These data show that cold shock conditions increased the transfection efficiency of mRNA, and consequently, nuclease-mediated genomic editing.

### Example 3: Increased Gene Editing Using ZFN

Bone marrow (BM) CD34+ cells were isolated from human bone marrow aspirates by first depleting erythrocytes, and then enriching CD34+ hematopoietic stem and progenitor cells (HSPC) using paramagnetic nanobead coupled CD34+ antibody with CliniMACS Prodigy (Miltenyi).

The initial electroporation of ZFN mRNA in BM-CD34+ HSPCs yielded low gene editing efficiency using a transfection protocol that has been optimized for CD34+ HSPCs from mobilized peripheral blood (PB-CD34+ ), however, high transfection efficiency (˜90%) was observed when the GFP mRNA was electroporated immediately after mixing with BM-CD34+ cells. Thus, the longer incubation of the ZFN mRNA with BM-CD34+ cells appeared to cause the lower gene editing efficiency. Without wishing to be bound to any theory, the mRNA may not be stable after mixing with the BM-CD34+ cells and become degraded before electroporation.

To solve this problem, an ice-chilling step was employed during the electroporation process. The following is the modified procedures for MaxCyte electroporation with OC-400, but the ice-chilling step can be applied to other scales

1. Prepare the washing buffer by adding final concentration of 0.1% human serum albumin (HSA) into MaxCyte EP buffer, keep the buffer on ice.

2. Cool down the Processing Chamber (i.e. OC-400, MaxCyte) in ice without opening.

3. Culture the bone marrow CD34+ hematopoietic stem and progenitor cells (BM-CD34+ HSPC) in X-VIVO media containing glutamax and 3 cytokine cocktail [(Recombinant Human Stem Cell Factor (SCF), Recombinant Human Thrombopoeitin (TPO), and Recombinant Human Flt-3 Ligand (Flt-3L)].

5. Collect HSPC at 200 g for 7.5 min (4° C. centrifuge), aspirate the supernatant

6. Add ice cold washing buffer prepared at step 1 (100× volume of the cell pellet). Spin HSPC at 200 g for 7.5 min, remove the supernatant. Spin the cells again at 200 g for 1 min, and remove the residual supernatant.

8. Add MaxCyte EP buffer to make cell concentration around 6.25 e7/ml. Keep the cell suspension on ice.

9. Add ice-cooled mRNA (formulated in nuclease-free water) to a desired final concentration (ZFN mRNA at ˜300 μg/ml). The added volume of mRNA should be <20% of the total volume.

10. Transfer cell-mRNA mixture into ice-cooled OC-400 prepared at step 2. (perform the process as quickly as possible after mRNA is added to cells).

11. Slide OC-400 into MaxCyte GT system and run the electroporation program. Remove cells from OC-400 and transfer the cells to a well (2×10\hat{ }6 cells/mL).

12. Incubate cells in a 37° C. incubator for 20 min.

13. Collect the incubated cells and resuspend the cells into pre-warmed full medium according to step 3.

The ice-chilling step was also implemented with other electroporator (i.e. BTX, Amaxia nucleofector) for mitigating the stability of the mRNA within BM-CD34+ HSPCs.

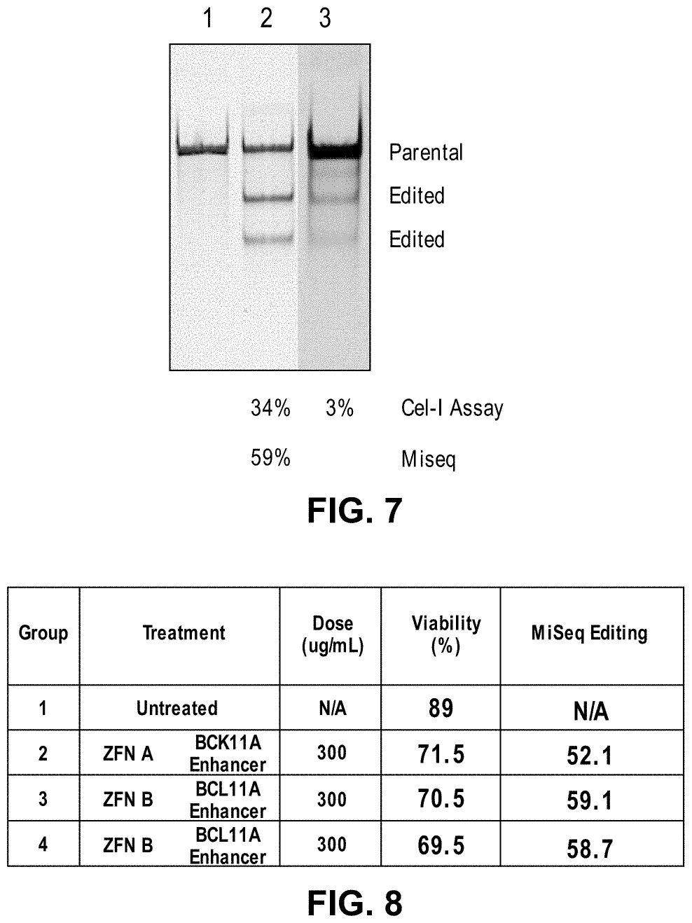

In the study of FIG. 7, bone marrow CD34+ cells were transfected with mRNA coding for zinc-finger nucleases either with (lane 2) or without (lane 3) the ice-chilling step. Lane 1 was un-transfected control. The gene editing level was measured by measured by the Cel-I assay. Ice-chilling during electroporation of mRNA increased the gene editing from about 3% (lane 3) to 34% (lane 2), indicating a ˜10 fold improvement in gene editing efficiency of zinc finger nucleases in bone marrow CD34+ cells.

In the study of FIG. 8, bone marrow CD34+ cells were transfected with mRNA coding for either ZFN A or ZFN B with the ice-chilling step as described above.

Both the ZFN A and ZFN B were designed to target to the BCL11A enhancer. Methods for making and using such ZFNs have been described, for example, in International Patent Publication No. WO 2015/073683. Gene editing efficiency was quantified by deep sequencing (MiSeq Editing). As indicated in FIG. 8, with the protocol that included an ice-chilling step, both ZFN A and ZFN B is capable of driving high efficient gene editing in bone marrow CD34+ cells without causing much cellular toxicity (i.e. viability ˜70%).

All patents, patent applications and publications mentioned herein are hereby incorporated by reference in their entirety.

Although disclosure has been provided in some detail by way of illustration and example for the purposes of clarity of understanding, it will be apparent to those skilled in the art that various changes and modifications can be practiced without departing from the spirit or scope of the disclosure. Accordingly, the foregoing descriptions and examples should not be construed as limiting.

What is claimed is:

1. A method for modifying hematopoietic stem cells and progenitor stem cells, the method comprising:
(a) cooling the cells in a vessel to about 15° C. or below; and
(b) introducing an exogenous RNA into the cooled cells under conditions such that the cell is modified by the introduction of the RNA.

2. The method of claim 1, wherein the method further comprises the step of cooling the vessel to about 15° C. or below.

3. The method of claim 2, wherein the method further comprises the step of contacting the cells with the cooled vessel to cool the cells.

4. The method of claim 1, wherein the method further comprises the step of cooling the exogenous RNA to about 15° C. or below.

5. The method of claim 4, wherein the method further comprises the step of combining the cooled exogenous RNA and cells within the vessel and maintaining the temperature of the vessel at about 15° C. or below.

6. The method of claim 1, wherein the method further comprises the step of placing the vessel in a device suitable for introducing the exogenous RNA into the cells.

7. The method of claim 6, wherein the device is an electroporation device.

8. The method of claim 6, wherein at least part of the device is cooled to about 15° C. or below prior to placing the vessel in the device.

9. The method of claim 8, wherein the part of the device cooled to about 15° C. or below operably contacts the vessel.

10. The method of claim 6, further comprising the step of incubating the cells under conditions suitable for expressing the RNA in the cell and modifying the cell.

11. The method of claim 1, wherein the exogenous RNA is mRNA, siRNA, sgRNA, RNAi and/or miRNA.

12. The method of claim 11, wherein the exogenous mRNA encodes a heterologous nuclease.

13. The method of claim 12, further comprising introducing a donor polynucleotide into the cell such that the donor polynucleotide is integrated into the genome of the cell.

14. The method of claim 12, wherein the heterologous nuclease is selected from the group consisting of a zinc finger nuclease (ZFN), a TALE-effector domain nuclease (TALEN) and/or a CRISPR/Cas nuclease system.

15. The method of claim 1, wherein the cells are bone marrow (BM)-derived CD34+ cells.

16. The method of claim 1, wherein the RNA encodes a zinc finger nuclease and the method further comprises the step of
(c) culturing the cells of step (b) at 27° C. to 33° C.; and
(d) culturing the cells of step (c) at an optimal growth temperature.

17. A cell made by the method of claim 1.
